(19) United States
(12) Patent Application Publication
(10) Pub. No.: US 2006/0078622 A1
(43) Pub. Date: Apr. 13, 2006

(54) Pharmaceutical formulations containing microparticles or nanoparticles of a delivery agent

(75) Inventors: Shingai Majuru, Brewster, NY (US); Puchun Liu, Somers, NY (US); Steve Dinh, Briarcliff Manor, NY (US); Jun Liao, Yorktown Heights, NY (US); Jongbin Lee, New City, NY (US); Ehud Arbit, Englewood, NJ (US); Nikhil Dhoot, Wappingers Falls, NY (US); Halina Levchik, Croton on Hudson, NY (US); George Klcin, Tarrytown, NY (US); Jamila Harris, Flushing, NY (US); Nai Fang Wang, Long Island City, NY (US)

Correspondence Address: DARBY & DARBY P.C., P.O. BOX 5257, NEW YORK, NY, 10150-5257, US

(73) Assignee: Emisphere Technologies, Inc., Tarrytown, NY (US)

(21) Appl. No.: 11/204,756

(22) Filed: Aug. 15, 2005

Related U.S. Application Data
(60) Provisional application No. 60/601,258, filed on Aug. 13, 2004.
(60) Provisional application No. 60/612,810, filed on Sep. 23, 2004.

(51) Int. Cl. A61K 9/14

(52) U.S. Cl. 424/489

(57) ABSTRACT

This invention relates to microparticles and/or nanoparticles containing a delivery agent and/or an active agent. This invention also relates to pharmaceutical formulations and solid dosage forms, including controlled release solid dosage forms of active agent and a delivery agent.

This application claims the benefit of U.S. Provisional Application No. 60/612,810, filed Sep. 23, 2004, and U.S. Provisional Application No. 60/601,258, filed Aug. 13, 2004, both of which are hereby incorporated by reference.

### FIELD OF THE INVENTION

This invention relates to pharmaceutical formulations and methods for preparing the same.

### BACKGROUND OF THE INVENTION

There is a continuing need for improved oral delivery systems for drugs, such as insulin.

### SUMMARY OF THE INVENTION

The present invention relates to microparticles and/or nanoparticles for oral administration containing a delivery agent compound alone or a combination of a delivery agent compound and an active agent. Formulations containing these particles (and, for particles containing only a delivery agent compound, and an active agent) provide significantly greater bioavailability of the active agent with less variability than oral administration of a simple mixture of the delivery agent compound and active agent as a powder, tablet, or capsule. Without being bound by any particular theory, it is believed that in at least some embodiments, this improvement may be due to (1) the small size of the micro- or nano-particles which permits them to pass from the stomach, through the pylorus (which typically has a diameter of 1000-2000 μm), to the small intestine, where particle dissolution and delivery agent-mediated drug absorption is believed to best occur, and (2) the intimate contact between the delivery agent compound and active agent in the particles which ensures that the delivery agent compound is present with the active agent at the site of absorption. Because the micro- and nano-particles freely pass through the pylorus into the small intestine, unlike a conventional tablet or capsule which must first become dissolved into particles sufficiently small to do so, variations caused by tablet disintegration and gastric transit modulated by gastric motility are minimized.

According to one embodiment, the particles comprising a delivery agent compound and an active agent have a median particle size less than about 900 or 1000 μm. For example, the median particle size can range from about 45 to about 850 μm, from about 45 to about 150 μm, from about 150 to about 250 μm, from about 250 to about 425 μm, from about 425 to about 850 μm, from about 100 to about 1000 nm, or from about 500 to about 1000 nm. According to another embodiment, the particles have a median particle size less than about 1 μm. In some embodiments, particles may be as small as about 1 nanometer and as large as about 999 micrometers. For example, the particles may have a median particle size of less than about 999 micrometers, from about 1 nanometer to about 999 micrometers, about 1 to about 999 micrometers, about 1 to about 999 nanometers, about 45 to about 850 micrometers, about 45 to about 150 micrometers, about 150 to about 250 micrometers, about 250 to about 425 micrometers, about 425 to about 850 micrometers, about 100 to about 1000 nanometers, or about 500 to about 1000 nanometers.

Another embodiment is a pharmaceutical formulation comprising a delivery agent compound and an active agent in which the delivery agent compound is in the form of particles. The particles can have a median particle size of less than about 999 micrometers, about 1 nanometer to about 999 micrometers, about 1 to about 999 nanometers, or about 7 to about 16 micrometers. Optionally, the active agent may also be in the form of particles. For example, the median particle size of the active agent particles may be less than about 999 micrometers, about 1 nanometer to about 999 micrometers, about 1 to about 999 micrometers, or about 1 to about 999 nanometers. According to one embodiment, the delivery agent particles and the active agent particles both have a median particle size of about 1 to about 999 micrometers. According to another embodiment, the delivery agent particles and the active agent particles both have a median particle size of about 1 to about 999 nanometers.

Yet another embodiment is a pharmaceutical formulation comprising a delivery agent and an active agent in which the active agent is in the form of particles having a median particle size of less than about 999 micrometers. According to one embodiment, the median particle size of the active agent particles is about 1 nanometer to about 999 micrometers, about 1 to about 999 micrometers, or about 1 to about 999 nanometers.

The particles can be in the form of fine granules or micro-beads (e.g., beads having a round/ball shape and a diameter of about 0.2 mm to about 2.0 mm). The micro-beads may be formed by compression. In one embodiment, the pharmaceutical formulation includes micro-beads containing a delivery agent compound, which are coated with an active agent, such as insulin or heparin. The micro-beads may have a diameter ranging from about 0.2 mm to 2.0 mm.

The particles may also include a mucoadhesive, such as a cellulose derivative (e.g., CMC sodium (available from Aqualon of Wilmington, Del.)) or a polyacrylic acid (e.g., Carbopol™ available from B.F. Goodrich of Cleveland, Ohio). The mucoadhesive can (1) facilitate adhesion to mucosa (including in the gastrointestinal tract) thereby prolonging delivery agent-active agent contact with the mucosa, (2) stabilize and protect the active agent (e.g., in the case of insulin), and (3) increase the permeability of biomembranes (including mucosa) thereby improving delivery and increasing bioavailability of the active agent.

It has also been discovered that oral administration of insulin in conjunction with a delivery agent compound by solid oral dosage forms that do not degrade in the stomach, but do degrade in the intestine, provides significantly greater bioavailability of the insulin. Such solid oral dosage forms containing insulin or a different active agent provide greater bioavailability than forms that degrade in the stomach and forms that do not contain the delivery agent compound. Without being bound by any particular theory, it is believed that this improvement is due to the sensitivity of insulin and other active agents to degradation by enzymes or acid found in gastric fluid. Because the solid oral dosage forms do not degrade in the stomach, the insulin and other active agents are protected from degradation until they reach the intestine.

Another embodiment of the invention is a pharmaceutical formulation (such as a solid oral dosage form) comprising a therapeutically effective amount of an active agent and a delivery agent, where the pharmaceutical formulation has a disintegration time of about 250 seconds to about 650 seconds when orally administered. In another embodiment, the disintegration time is about 350 to about 550 seconds when orally administered. In yet another embodiment, the disintegration time is greater than 60 seconds when orally administered. In yet another embodiment, the disintegration time is greater than 400 seconds when orally administered. Disintegration time can be determined in water at 37±2° C. using the method described in USP <701>. Disintegration times may range from about 1 second to as much as about 24 hours, or more, depending on many factors including, but not limited to, the particular active agent(s), delivery agent compound(s), and excipients included in the pharmaceutical formulation.

Another embodiment is a pharmaceutical formulation (such as a solid oral dosage form) comprising a therapeutically effective amount of an active agent and a delivery agent, where the solid oral dosage form does not substantially disintegrate or dissolve in the stomach, but does substantially disintegrate or dissolve in the intestine. In a preferred embodiment, the active agent is insulin. In another preferred embodiment, the active agent is an insulin derivative.

In another embodiment, the pharmaceutical formulation is a solid oral dosage form which is covered with an enteric coating to retard disintegration in the stomach. Enteric coatings include, but are not limited to, hydroxypropyl methylcellulose phthalate, hydroxypropyl methylcellulose acetate succinate, polyvinyl acetate phthalate, cellulose acetate trimellitate, cellulose acetate phthalate, poly(methacrylic acid-ethylacrylate), and poly(methacrylic acid-methyl methacrylate).

In yet another embodiment, the pharmaceutical formulations may be formulated to erode from the surface of the dosage form, rather than disintegrate.

The pharmaceutical formulations may include enzyme-inhibiting agents to prevent enzymatic degradation of active agents in the pharmaceutical formulation.

In one embodiment, the delivery agent is a compound having the following structure or a salt thereof: 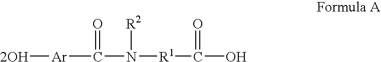
wherein

Ar is phenyl or naphthyl;

Ar is optionally substituted with one or more of —OH, halogen, C₁-C₄ alkyl, C₁-C₄ alkenyl, C₁-C₄ alkoxy. or C₁-C₄ haloalkoxy;

R¹ is C₃-C₂₀ alkyl, C₄-C₂₀ alkenyl, phenyl, naphthyl, (C₁-C₁₀ alkyl) phenyl, (C₁-C₁₀ alkenyl)phenyl, (C₁-C₁₀ alkyl)naphthyl, (C₁-C₁₀ alkenyl) naphthyl, phenyl(C₁-C₁₀ alkyl), phenyl(C₁-C₁₀ alkenyl), naphthyl(C₁-C₁₀ alkyl), or naphthyl(C₁-C₁₀ alkenyl);

R¹ is optionally substituted with C₁ to C₄ alkyl, C₂ to C₄ alkenyl, C₁ to C₄ alkoxy, C₁-C₄ haloalkoxy, —OH, —SH, —CO₂R⁸, or any combination thereof;

R² is hydrogen, C₁ to C₄ alkyl, or C₂ to C₄ alkenyl; and

R¹ is optionally interrupted by oxygen, nitrogen, sulfur or any combination thereof. The term “2-OH—Ar” in formula A refers to a phenyl or naphthyl group having a hydroxyl group at the 2-position.

According to one embodiment, the compounds are not substituted with an amino group in the position alpha to the acid group.

Preferably, Ar is substituted with a halogen.

Preferably, R² is hydrogen.

Preferably, R¹ is unsubstituted.

Preferably, R¹ is not interrupted.

Preferably, R¹ is C₁₋₁₀, C₃₋₉, C₃₋₇, C₃, C₇, or C₉ alkyl. According to one embodiment, R¹ is not branched.

Preferred delivery agent compounds include, but are not limited to, N-(8-[2-hydroxybenzoyl]amino)caprylic acid (the free acid of SNAC), N-(10-[2-hydroxybenzoyl]amino)decanoic acid (the free acid of SNAC), 4-[(2-hydroxy-4-chloro-benzoyl)-amino]butanoic acid (the free acid of 4-CNAB), and salts thereof, and solvates and hydrates thereof. The salt can be, for example, a sodium salt, such as a monosodium (i.e., SNAC, SNAD, or 4-CNAB) or disodium salt.

In another embodiment, the delivery agent is a compound having the following structure or a salt thereof: 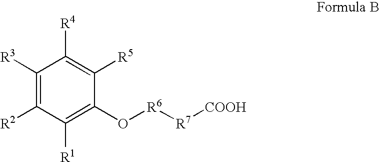
wherein

R¹, R², R³, and R⁴ are independently H, —OH, halogen, C₁-C₄ alkyl, C₂-C₄ alkenyl, C₁-C₄ alkoxy, —C(O)R⁸, —NO₂, —NR⁹R¹⁰, or —N⁺R⁹R¹⁰R¹¹(R¹²)⁻;

R⁵ is H, —OH, —NO₂, halogen, —CF₃, —NR¹⁴R¹⁵, —N⁺R¹⁴R¹⁵R¹⁶(R¹³)⁻, amide, C₁-C₁₂ alkoxy, C₁-C₁₂ alkyl, C₂-C₁₂ alkenyl, carbamate, carbonate, urea, or —C(O)R¹⁸;

R⁵ is optionally substituted with halogen, —OH, —SH, or —COOH;

R⁵ is optionally interrupted by O, N, S, or —C(O)—;

R⁶ is a C₁-C₁₂ alkylene, C₂-C₁₂ alkenylene, or arylene;

R⁶ is optionally substituted with a C₁-C₄ alkyl, C₂-C₄ alkenyl, C₁-C₄ alkoxy, —OH, —SH, halogen, —NH₂, or —CO₂R⁸;

R⁶ is optionally interrupted by O or N;

R⁷ is a bond or arylene;

R⁷ is optionally substituted with —OH, halogen, —C(O)CH₃, —NR¹⁰OR¹¹, or —N⁺R¹⁰R¹¹R¹²(R¹³)⁻;

R⁸ is H, C₁-C₄ alkyl, C₂-C₄ alkenyl, or —NH₂;

R⁹, R¹⁰, R¹¹, and R¹² are independently H or C₁-C₁₀ alkyl;

R¹³ is a halide, hydroxide, sulfate, tetrafluoroborate, or phosphate;

R¹⁴, R¹⁵, and R¹⁶ are independently H, C₁-C₁₀ alkyl, C₁-C₁₀ alkyl substituted with —COOH, C₂-C₁₂ alkenyl, C₂-C₁₂ alkenyl substituted with —COOH, or —C(O)R¹⁷;

R¹⁷ is —OH, C₁-C₁₀ alkyl, or C₂-C₁₂ alkenyl; and

R¹⁸ is H, C₁-C₆ alkyl, —OH, —NR¹⁴R¹⁵, or N⁺R¹⁴R¹⁵R¹⁶ (R¹³)⁻.

In yet another embodiment, the delivery agent is a compound having the following structure or a salt thereof: 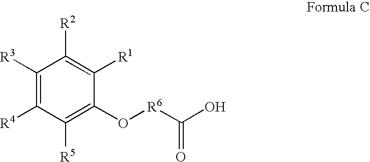
wherein

R¹, R², R³, R⁴ and R⁵ are independently H, —CN, —OH, —OCH₃, or halogen, at least one of R¹, R², R³, R⁴ and R⁵ being —CN; and

R⁶ is a C₁-C₁₂ linear or branched alkylene, alkenylene, arylene, alkyl(arylene) or aryl(alkylene).

In yet another embodiment, the delivery agent is a compound having the following structure or a salt thereof: 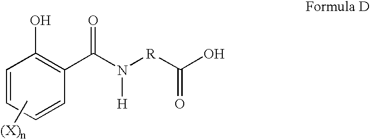
wherein

each occurrence of X is hydrogen, halogen, hydroxyl, or C₁-C₃ alkoxy,

R is substituted or unsubstituted C₁-C₃ alkylene or substituted or unsubstituted C₂-C₃ alkenylene, and

n is an integer from 1 to 4.

In yet another embodiment, the delivery agent is a compound having the following structure or a salt thereof: 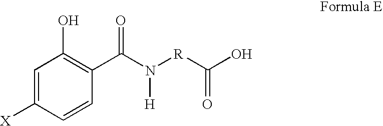
wherein

X is halogen, and R is substituted or unsubstituted C₁-C₃ alkylene or substituted or unsubstituted C₂-C₃ alkenylene.

Preferred delivery agent compounds include but are not limited to, N-(8-[2-hydroxybenzoyl]-amino)caprylic acid, N-(10-[2-hydroxybenzoyl]-amino)decanoic acid, 8-(2-hydroxy-4-methoxybenzoylamino)octanoic acid, 8-(2-hydroxy-5-chlorobenzoylamino)-octanoic acid, 4-[(2-hydroxy-4-chlorobenzoyl)amino]butanoic acid, and pharmaceutically acceptable salts thereof. The pharmaceutical formulations of the present invention may include any of the aforementioned delivery agent compounds, or any other delivery agent compounds, alone or in combination with one or more additional delivery agent compounds.

Suitable active agents include but are not limited to, proteins, polypeptides, peptides, hormones, polysaccharides, as well as synthetic, natural or recombinant sources thereof: growth hormones; growth hormone releasing hormones; growth hormone releasing factor, interferons; interleukin-1; interleukin-2; insulin, optionally having counter ions including zinc, sodium, calcium and ammonium; insulin-like growth factor; heparin; calcitonin; erythropoietin; atrial naturetic factor; antigens; monoclonal antibodies; somatostatin; protease inhibitors; adrenocorticotropin, gonadotropin releasing hormone; oxytocin; leutinizing-hormone-releasing-hormone; follicle stimulating hormone; glucocerebrosidase; thrombopoietin; filgrastim; prostaglandins; cyclosporin; vasopressin; cromolyn sodium; vancomycin; desferrioxamine; bisphosphonates; parathyroid hormone; anti-migraine agents; glucagon-like peptide 1 (GLP-1); antimicrobials; vitamins; and analogs, fragments, mimetics or polyethylene glycol (PEG)-modified derivatives of these compounds; or any combination thereof. Preferred active agents include, but are not limited to, insulin and heparin (including, but not limited to, unfractionated heparin and low molecular weight heparin).

In one embodiment of the present invention, the active agent is insulin. The insulin-containing pharmacuetical formulations of the present invention may also include a second hypoglycemic agent, an inhibitor of renal glucose reabsorption, or any combination of the foregoing (such as those described in U.S. Patent Publication No. 2005/0143424, which is hereby incorporated by reference). Suitable second hypoglycemic agents include, but are not limited to, insulin secretion-promoting agents, insulin resistance-ameliorating agents, insulin mimetics, α-glucosidase inhibitors, glucogenesis inhibitors, and any combination of any of the foregoing. According to one embodiment, the solid dosage form includes a sulfonyl urea, meglitinide analogue, biguanide (preferably metformin), or any combination of any of the foregoing. According to a preferred embodiment, the solid dosage form includes metformin.

Also provided is a pharmaceutical formulation, such as a solid dosage unit form, comprising the microparticles or nanoparticles of the present invention and/or having the disintegration times discussed above. The dosage unit form may be in the form of a tablet, capsule, powder, or sachet. The dosage unit form may have, alone or in combination, one or more enteric coatings, disintegrants, super disintegrants (such as sodium starch glycolate or croscarmellose sodium), and extra particle super disintegrants.

In one embodiment, the solid oral dosage unit form is a fast disintegrating tablet. In another embodiment, the solid dosage unit form has a controlled or delayed release.

According to one embodiment, the present invention provides a tablet comprising the aforementioned particles and a disintegrant. In one embodiment, the disintegrant is a super disintegrant, such as sodium starch glycolate (Primojel® available from Azebe UK Ltd. of South Humberside, UK), croscarmellose sodium (Primellose® available from Azebe UK Ltd. of South Humberside, UK), or an extra particle super disintegrant.

Another embodiment is a solid dosage form comprising a therapeutically effective amount of insulin and a delivery agent compound, where the solid dosage form has a disintegration time of at least 60 seconds when administered orally. The solid dosage form may have an enteric coating or be a surface eroding formulation. The solid dosage form may further comprise one or more enzyme inhibiting agents.

Yet another embodiment is a solid dosage form comprising a therapeutically effective amount of insulin and a delivery agent compound, where the solid dosage form does not substantially disintegrate or dissolve in the stomach but does disintegrate or dissolve in the small intestine. The solid dosage form may have an enteric coating or be a surface eroding formulation. The solid dosage form may further comprise one or more enzyme inhibiting agents.

Another embodiment is a method for administering an active agent to an animal, particularly an animal in need of the active agent, by administering a pharmaceutical formulation comprising the microparticles or nanoparticles of the present invention and/or those having the disintegration times discussed above (i.e. those having a controlled or sustained release). Oral administration is a preferred route of administration.

Yet another embodiment is a method of treating a disease or for achieving a desired physiological effect in an animal by administering a pharmaceutical formulation of the present invention, including solid unit dosage forms comprising the microparticles or nanoparticles of the present invention and/or those having the disintegration times discussed above (i.e. those having a controlled or sustained release). Yet another embodiment is a method of increasing the oral bioavailability of active agents by orally administering a pharmaceutical formulation of the present invention.

Yet another embodiment is a method of treating diabetes and/or reducing the incidence of systemic hyperinsulinemia associated with chronic dosing of insulin in a mammal (such as in a human, particularly a human in need thereof) by administering to the mammal a therapeutic effective amount of an insulin-containing pharmaceutical formulation of the present invention, e.g., those comprising the microparticles or nanoparticles of the present invention and/or those having the disintegration times discussed above. In one embodiment, the delivery agent compound is the free acid of 4-CNAB or a pharmaceutically acceptable salt thereof. The pharmaceutical formulation may be administered on a chronic basis.

Yet another embodiment is a method of treating impaired glucose tolerance, early stage diabetes, or late stage diabetes or achieving glucose homeostasis in a mammal (such as in a human, particularly in need thereof) by administering to the mammal a therapeutic effective amount of an insulin-containing pharmaceutical formulation of the present invention, such as a pharmaceutical formulation comprising the microparticles or nanoparticles of the present invention and/or having the disintegration times discussed above. In one embodiment, the delivery agent compound is the free acid of 4-CNAB or a pharmaceutically acceptable salt thereof. The pharmaceutical formulation may be administered on a chronic basis.

Yet another embodiment is a method of treating a human diabetic patient by orally administering to the human diabetic patient on a chronic basis a therapeutic effective amount of an insulin-containing pharmaceutical formulation described herein.

Yet another embodiment is a method of preparing the micro- and nano-particles of the present invention by drying a solution of a delivery agent compound and an active agent, for example, until a solid is formed, and optionally, isolating the particles. Preferably, the mixture is homogenous (e.g., the delivery agent compound and the active agent are uniformly distributed throughout the mixture). The method includes co-drying a mixture of the delivery agent compound, the active agent, and a solvent. Suitable solvents include, but are not limited to, hydroxylic solvents, water, and mixtures thereof. According to one embodiment, the mixture is dried at from about 10 to about 40° C. (e.g., at room temperature). Preferably, the drying is performed at a controlled temperature. According to one embodiment, the drying is performed over an inert gas (preferably nitrogen gas). The dried material may optionally be milled and/or sieved to obtain the desired particle size. This method results in particles containing a homogeneous mixture of the delivery agent compound and the active agent.

Another method of preparing the micro- and nano-particles of the present invention is by lyophilizing a mixture of the delivery agent compound, the active agent, and a solvent. Suitable solvents include, but are not limited to, hydroxylic solvents, water, and mixtures thereof.

Yet another method of preparing the micro- and nano-particles of the present invention is by (1) dissolving a delivery agent compound and an active agent in a supercritical fluid, and (2) decreasing the system pressure to deposit the delivery agent compound and active agent as extremely fine particles. The deposition is a result of the rapid expansion of the supercritical solution.

The following embodiments are collectively referred to herein as the “solid pharmaceutical composition embodiments”.

Yet another embodiment is a solid pharmaceutical composition which enhances the oral bioavailability of active agents, particularly peptides. More specifically, the solid pharmaceutical composition suitable for the oral delivery of pharmacologically active agents, comprises:
1. a therapeutically-effective amount of a pharmacologically active agent;
2. one or more pharmaceutically acceptable inactive excipients; and
3. a delivery agent for the pharmacologically active agent, wherein the delivery agent is in micronized form.

Yet another embodiment is a solid pharmaceutical composition suitable for the oral delivery of calcitonin, comprising:
1. a therapeutical-effective amount of a calcitonin; and
2. one or more pharmaceutically acceptable inactive excipients, and
3. a delivery agent for said calcitonin, wherein said delivery agent is in micronized form.

In an additional embodiment, the pharmaceutically acceptable inactive excipient may be either or both of the polymers crospovidone or povidone.

In a still further embodiment, the solid pharmaceutical composition suitable for oral delivery may also comprise a diluent.

In addition, in another embodiment the solid pharmaceutical composition suitable for oral delivery may also comprise a lubricant.

In a further embodiment, the invention is directed to a method for enhancing the oral bioavailability of a pharmacologically active agent. The method comprises administering to a subject in need of the pharmacologically active agent an effective amount of a pharmaceutical composition according to the instant invention.

Yet another embodiment is a method of treatment of bone related diseases and calcium disorders comprising administering to a patient in need of such treatment a therapeutically effective amount of a composition according to the instant invention, wherein the pharmacologically active agent is calcitonin.

The above features and many other attendant advantages of the invention will become better understood by reference to the following detailed description when taken in conjunction with the accompanying drawings.

### BRIEF DESCRIPTION OF THE DRAWINGS

FIG. 1 depicts a schematic of direct dosing to the stomach and the jejunum.

FIG. 2 is a graph of the concentration of insulin level (±SEM) following direct dosing of coprocessed microparticles to the stomach and the jejunum over time.

FIG. 3 is a graph of the change in glucose level (±SEM) following direct dosing of coprocessed microparticles to the stomach and the jejunum over time.

FIG. 4 is a graph of the change in glucose (±SEM) following oral gavage from 3 different dosage forms: 1) a tablet made by compressing insulin and carrier, 2) a capsule containing microparticles of coprocessed insulin and carrier, and 3) a capsule containing a simple mixture of insulin and carrier, over time.

FIG. 5 is a graph of the insulin level (±SEM) following oral gavage from 3 different dosage forms: 1) a tablet made by compressing insulin and carrier, 2) a capsule containing microparticles of coprocessed insulin and carrier, and 3) a capsule containing a simple mixture of insulin and carrier, over time.

FIG. 6 is a graph of the insulin level (±SEM) following oral gavage of a capsule containing microparticles of coprocessed insulin and carrier over time. Two of the ten rats exhibited significantly high insulin absorption. The average values with (N=0) and without (N=8) inclusion of these two high responders are depicted in the graph.

FIG. 7 is a graph of the change in glucose (±SEM) following oral gavage of a capsule containing microparticles of coprocessed insulin and carrier over time. Two of the ten rats exhibited significantly high insulin absorption. The average values with (N=10) and without (N=8) inclusion of these two high responders are depicted in the graph.

FIG. 8 is a chart of the estimated absolute bioavailability (±SEM) from in situ dosing of coprocessed insulin and carrier to the stomach and the jejunum. Two compositions were evaluated: 1) insulin (0.25 mg/kg)+delivery agent (37.5 mg/kg), and 2) insulin (0.5 mg/kg)+delivery agent (75 mg/kg).

FIG. 9 is a chart of the estimated absolute bioavailability of insulin level (±SEM) from 1) subcutaneous administration, 2) direct dosing to the stomach, 3) direct dosing to the jejunum, 4) a tablet made by compressing insulin and carrier, 5) a capsule containing microparticles of coprocessed insulin and carrier with and without inclusion of the two high responders, and 6) a capsule containing a simple mixture of insulin and carrier.

FIG. 10 is a chart of the estimated bioavailability of insulin in the portal vein (±SEM) from 1) direct dosing to the stomach, 2) direct dosing to the jejunum, 3) a tablet made by compressing insulin and carrier, 4) a capsule containing microparticles of coprocessed insulin and carrier with and without inclusion of the two high responders, and 5) a capsule containing a simple mixture of insulin and carrier.

FIG. 11 is a chart of the estimated bioavailability (±SEM) of insulin relative to subcutaneous administration from 1) direct dosing to the stomach, 2) direct dosing to the jejunum, 3) a tablet made by compressing insulin and carrier, 4) a capsule containing microparticles of coprocessed insulin and carrier with and without inclusion of the two high responders, and 5) a capsule containing a simple mixture of insulin and carrier.

FIG. 12 is a chart of the estimated bioavailability of insulin in the portal vein relative to subcutaneous administration (±SEM) from 1) direct dosing to the stomach, 2) direct dosing to the jejunum, 3) a tablet made by compressing insulin and carrier, 4) a capsule containing microparticles of coprocessed insulin and carrier with and without inclusion of the two high responders, and 5) a capsule containing a simple mixture of insulin and carrier.

FIG. 13 is a graph of the individual insulin levels following oral gavage of a capsule containing microparticles of coprocessed insulin and carrier over time. Rat 14 and rat 17 exhibited significantly high insulin absorption. The average values with (N=10) and without (N=8) inclusion of these two high responders are depicted in the graph.

FIG. 14 is a graph of the individual glucose change following oral gavage of a capsule containing microparticles of coprocessed insulin and carrier over time. Rat 14 and rat 17 exhibited significantly high insulin absorption. The average values with (N=10) and without (N=8) inclusion of these two high responders are depicted in the graph.

FIG. 15 is a graph of the individual insulin level following oral gavage of a capsule containing microparticles of coprocessed insulin and carrier over time. Rats 14 and 17 were omitted. The average value from N=8 is depicted in the graph.

FIG. 16 is a graph of the individual glucose change following oral gavage of a capsule containing microparticles of coprocessed insulin and carrier over time. Rats 14 and 17 were omitted. The average value from N=8 is depicted in the graph.

FIG. 17 is a graph depicting the changes over time in serum glucose levels in rhesus monkeys that have been fed formulations 1-6, described below, containing insulin and a delivery agent. These formulations have varying disintegration times.

FIG. 18 is a graph depicting the changes over time in serum insulin concentration rhesus monkeys that have been fed formulations 1-6, described below, containing insulin and a delivery agent. These formulations have varying disintegration times.

FIG. 19 is a graph of anti-factor Xa activity (U/ml) versus time in monkeys after administration of the SNAD/heparin formulation described in Example 10.

### DETAILED DESCRIPTION OF THE INVENTION

### DEFINITIONS

The “particles,” “micro-beads,” and “granules” described herein may be any shape and can include one or more ingredients in addition to the delivery agent compound and/or active agent. The specific ingredients of any given particle, micro-bead, or granule, may also depend on the processes used and will not necessarily be the same in each individual particle, micro-bead, or granule from a batch.

For example, where particles, micro-beads, or granules of an active agent are prepared separately from particles, micro-beads, or granules of a delivery agent compound, the active agent particles, micro-beads, or granules will, generally, not comprise delivery agent compound, and the delivery agent particles, micro-beads, or granules will, generally, not comprise active agent, though each particle, micro-bead, or granule may comprise other ingredients, as disclosed herein.

In other embodiments, particles, micro-beads, or granules may be formed from a solution, suspension or mixture, in liquid or dry form, without limitation, which comprises at least an active agent and a delivery agent compound. Thus, for example, any given particle, micro-bead, or granule comprises both active agent and delivery agent compound, and may further comprise one or more other ingredients.

The terms “diameter” and “median particle size” are generally used to refer to the dimensions of particles, micro-beads, and granules. The “median particle size” or “diameter” was determined as follows for Examples 8, 9, 10.

Instrument: Mastersizer 2000 (EQ 202, model MS2K, serial number 34315-67)

Manufacturer: MALVERN instruments, England

Software: Mastersizer 2000

Accessory: Scirocco 2000 (A) (model ADA 2000, serial number 34270/73)

Dispersant: Dry dispersion

Analysis model: General purpose

Particle RI: 1.520

Obscuration: 1-6%

Standards: Malvern Quality Audit Standard for Sample Dispersion Units

The Malvern Mastersizer 2000 determines particle size by laser diffraction and model fitting. A well-dispersed sample in any two-phase system (e.g., powders, suspensions, or emulsions) is introduced into the path of a He—Ne laser focused with a lens of a length suitable for particle sizes present in the sample. The scattering pattern of particles in the laser path is measured by an array of detectors, with each detector measuring data from a particular range of angles.

The Malvern apparatus assumes that the particles being measured are perfect spheres. For non-spherical particles the resulting particle size distribution may be different from those obtained by methods based on others principles. The electronic measurements will often have to be accompanied by microscopic investigation to determine the type of particles being investigated. For irregularly shaped particles, the particle size data obtained from Mastersizer 2000 will be interpreted as the diameter of an imaginary sphere that is equivalent in volume to the measured particle. (Note: d(0.1) is the size of particle for which 10% of the sample is below this size, d(0.5) is the size of particle for which 50% of the sample is below this size, and d(0.9) is the size of particle for which 90% of the sample is below this size.

Generally, this apparatus measures one dimension of a, e.g., particle as it travels past a laser; i.e., it measures the length of a straight line through the particle. For irregular particles, this results in a variation of results since the orientation of a particle relative to the laser may result in the single measurement being taken of that individual particle's longest, shortest, or any other dimension. However, a measurement is taken of a number of particles and a median diameter or size is calculated. Thus, “size” or “diameter” figures are estimates of the median “size” or “diameter” of particles. Alternatively, “diameter” or “size” was measured by a sieve method described in Example 1. “Diameter” should not be read to necessarily imply a spherical shape or a circular dimension, though in certain embodiments, e.g., particles may have rounded edges or generally spherical shapes.

It should be understood, also, that the invention is not limited to particles, micro-beads, or granules which fall within a narrow range of “sizes” or “diameters”. Thus, for example, some embodiments may comprise, depending at least on the ingredients and processes used, some particles which fall within, for example, both the nanometer and micrometer scale, in the same batch. The actual “sizes” or “diameters” of the individual particles may fall within a relatively narrow or relatively large range.

As used herein and in the appended claims, the singular forms “a,” “an,” and “the,” include plural referents unless the context clearly indicates otherwise. Thus, for example, reference to “a particle” includes one or more of such particles, reference to “an” active agent includes one or more of such active agents, and “a” delivery agent includes one or more delivery agents,

The term “about” generally means within 10%, preferably within 5%, and more preferably within 1% of a given value or range.

The term “hydrate” as used herein includes, but is not limited to, (i) a substance containing water combined in the molecular form and (ii) a crystalline substance containing one or more molecules of water of crystallization or a crystalline material containing free water.

The term “solvate” as used herein includes, but is not limited to, a molecular or ionic complex of molecules or ions of a solvent with molecules or ions of the delivery agent compound or salt thereof, or hydrate or solvate thereof.

The term “delivery agent” refers to any of the delivery agent compounds disclosed herein.

The term “SNAC” refers to the monosodium salt of N-(8-[2-hydroxybenzoyl]-amino)caprylic acid, including the various polymorphic forms of the monosodium salt described in U.S. Provisional Application No. 60/569,476, filed May 6, 2004 (which is hereby incorporated by reference) unless otherwise indicated.

The term “SNAD” refers to the monosodium salt of N-(10-[2-hydroxybenzoyl]-amino)decanoic acid, unless otherwise indicated. The term “disodium salt of SNAD” refers to the disodium salt of N-(10-[2-hydroxybenzoyl]-amino)decanoic acid.

The term “5-CNAC” refers to the monosodium salt of N-(8-[2-hydroxy-5-chlorobenzoyl]-amino)octanoic acid, unless otherwise indicated.

The term “4-CNAB” refers to the monosodium salt of sodium N-4-[(2-hydroxy-4-chlorobenzoyl)amino]butanoate, including anhydrous, monohydrate, and isopropanol solvates thereof and various polymorphic forms of the monosodium salt described in International Publication No. WO 03/057650 (which is hereby incorporated by reference), unless otherwise indicated.

An “effective amount of active agent” is an amount of active agent which is effective to treat or prevent a condition in a living organism to whom it is administered over some period of time, e.g., provides a therapeutic effect during a desired dosing interval.

The term “insulin” refers to all forms of insulin, including, but not limited to, naturally derived insulin and synthetic forms of insulin, such as those described in U.S. Pat. Nos. 4,421,685, 5,474,978, and 5,534,488, each of which is hereby incorporated by reference in its entirety.

The term “insulin derivatives” refers to insulin-derived proteins and peptides with insulin actions, and include, for example, lispro, B10Asp and HOE-901.

An “effective amount of delivery agent” is an amount of the delivery agent which enables and/or facilitates the absorption of a desired amount of active agent via any route of administration (such as those discussed in this application including, but not limited to, the oral (e.g., across a biological membrane in the gastrointestinal tract), nasal, pulmonary, dermal, buccal, vaginal, and/or ocular route).

The terms “alkyl” and “alkenyl” as used herein include linear and branched alkyl and alkenyl substituents, respectively.

The phrase “pharmaceutically acceptable” refers to additives or compositions that are physiologically tolerable when administered to a mammal.

The phrase “substantially disintegrate” means that about75%toabout 95% of the total volume of the tablet will break apart and dissolve into its component parts (e.g. insoluble coated particles, insoluble disintegrant, etc.), and the tablet is no longer intact except for small aggregates.

“Surface eroding formulation” refers to formulations that do not disintegrate but instead erode, e.g., the formulation dissolves from the surface over a pre-determined period of time and the tablet generally remains intact and retains its overall shape. The surface eroding formulations allow for sustained release of an active agent over the pre-determined time period.

The terms “micronize” and “micronized” generally refer to a process, or particles which have been processed, such that their diameters/sizes are within the general range of microparticles and/or nanoparticles.

The term “microparticle” generally includes particles having a diameter ranging from about 1 to about 999 micrometers (microns, μm).

The term “nanoparticle” generally includes particles having a diameter ranging from about 1 to about 999 nanometers (nm).

The term “insulin derivatives” includes insulin-derived proteins and peptides with insulin actions, and include, for example, lispro, B10Asp and HOE-901.

“Insulin secretion-promoting agents” exert their hypoglycemic action, by mainly influencing pancreatic β-cells to promote insulin secretion into blood, and include, for example, sulfonylureas (for example, tolbutamide, chlorpropamide, glibenclamide (glyburide), glipizide, glimeperide, and gliclazide); and meglitinide analogues (for example, repaglinide, nateglinide, meglitinide and mitiglinide (KAD-1229))). Other insulin secretion-promoting agents are, for example, K⁺-ATP channel inhibitors (for example, BTS-67-582), glucagon-like peptide-1 receptor agonists (for example, glucagon-like peptide-1, exendin-4 and NN-2211) and dipeptidyl peptidase-IV inhibitors with an effect of enhancing the action of glucagon-like peptide-1. According one embodiment, the insulin secretion-promoting agent is a sulfonylurea or meglitinide analogue.

The term “insulin resistance-ameliorating agents” includes agents exerting hypoglycemic action by enhancing the action of insulin in target tissues, and include for example peroxisome proliferator activator receptor (PPAR)-γ agonists (for example, thiazolidine-based compounds such as pioglitazone, rosiglitazone, and ciglitazone; or non-thiazolidine-based compounds such as GI-262570, JTT-501, YM-440, NN-622 and KRP-297), PPAR-γ antagonists and protein tyrosine phosphatase inhibitors. The insulin resistance-ameliorating agents include, for example, pharmaceutical agents with a function ameliorating insulin resistance, for example biguanides (for example, metformin, phenformin and buformin, preferably metformin), PPAR-α agonists (fibrate-series compounds such as simfibrate, clofibrate, bezafibrate and clinofibrate and non-fibrate-series compounds), anti-obesity agents (for example, 5-hydroxytryptamine (5-HT) reuptake inhibitors such as sibutramine, lipase inhibitors such as orlistat and adrenalin β-receptor agonists such as AJ-9677). Preferred insulin resistance-ameliorating agents include, but are not limited to, biguanides, such as metformin.

The term “insulin mimetics” refers to agents expressing the hypoglycemic action through physiological insulin action, namely the action promoting glucose uptake into cells, in a manner more or less independent to insulin, except for insulin derivatives, and include for example insulin receptor-activating agents (for example, CLX-0901 and L-783281) and vanadium.

The term “α-glucosidase inhibitors” refers to agents expressing the hypoglycemic action through suppression of glucose absorption into bodies, mainly via the inhibition of α-glucosidase in the intestinal tube and include, for example, acarbose, voglibose and miglitol.

The term “glucogenesis inhibitors” refers to agents expressing hypoglycemic action mainly through the inhibition of glucogenesis, and include for example glucagon secretion suppressors (for example, M&B-39890A and octreotide), fatty acid decomposition inhibitors (for example, nicotinic acid derivatives and camitine palmitoyltransferase-1 inhibitor) and glucose-6-phosphatase inhibitors.

The term “inhibitor of renal glucose reabsorption” refers to agents which inhibit glucose reabsorption in uriniferous tubules. The primary action of the inhibitor of renal glucose reabsorption is not involved in the promotion of the uptake into target tissue cells, the suppression of the absorption from intestinal tube, or the hypoglycemic action via the suppression of the synthesis in tissues. Suitable inhibitors of renal glucose reabsorption include, but are not limited to, those described in U.S. Patent Publication No. 2005/0143424, which is hereby incorporated by reference.

Delivery Agent Compounds

The delivery agent compound may be any of those described in U.S. Pat. Nos. 5,650,386 and 5,866,536 and International Publication Nos. WO94/23767, WO95/11690, WO95/28920, WO95/28838, WO96/10396, WO96/09813, WO96/12473, WO96/12475, WO96/30036, WO96/33699, WO97/31938, WO97/36480, WO98/21951, WO98/25589, WO98/34632, WO98/49135, WO99/16427, WO0/06534, WO00/07979, WO00/40203, WO00/46182, WO00/47188, WO00/48589, WO00/50386, WO00/59863, WO00/59480, WO01/32130, WO01/32596, WO01/34114, WO01/44199, WO01/51454, WO01/70219, WO01/92206, WO02/02509, WO02/15959, WO02/16309, WO02/20466, W002/19969, WO02/070438, WO03/026582, WO02/100338, WO03/045306, WO03/26582, and WO 03/057170, all of which are hereby incorporated by reference.

Non-limiting examples of delivery agent compounds include N-(8-[2-hydroxybenzoyl]amino)caprylic acid, N-(10-[2-hydroxybenzoyl]amino)decanoic acid, 8-(2-hydroxy-4-methoxybenzoylamino)octanoic acid, 8-(2-hydroxy-5-chlorobenzoyl-amino)octanoic acid, 4-[(2-hydroxy-4-chlorobenzoyl)amino]butanoic acid, and salts thereof. Preferred salts include, but are not limited to, monosodium and disodium salts.

According to one embodiment, the delivery agent compound is N-(8-[2-hydroxybenzoyl]amino)caprylic acid or a pharmaceutically acceptable salt thereof.

According to another embodiment, the delivery agent compound is N-(10-[2-hydroxybenzoyl]amino)decanoic acid or a pharmaceutically acceptable salt thereof.

According to another embodiment, the delivery agent compound is 4-[(2-hydroxy-4-chlorobenzoyl)amino]butanoic acid or a pharmaceutically acceptable salt thereof.

According to another embodiment, the delivery agent compound is 8-(2-hydroxy-5-chlorobenzoylamino)octanoic acid or a pharmaceutically acceptable salt thereof.

The delivery agent compounds may be in the form of the carboxylic acid or pharmaceutically acceptable salts thereof, such as sodium salts, and hydrates and solvates thereof. The salts may be mono- or multi-valent salts, such as monosodium salts and disodium salts (e.g., the disodium salt of 8-(2-hydroxy-5-chlorobenzoylamino)-octanoic acid, the disodium salt of N-(8-[2-hydroxybenzoyl]amino)caprylic acid, the disodium salt of N-(10-[2-hydroxybenzoyl]amino)decanoic acid). See, for example, International Publication No. WO 00/59863, which is hereby incorporated by reference The delivery agent compounds may contain different counter ions chosen for example due to their effect on modifying the dissolution profile of the carrier.

The delivery agent compounds may be prepared by methods known in the art, such as those discussed in the aforementioned publications (e.g., International Publication Nos. WO 98/34632, WO 00/07979, WO 01/44199, WO 01/32596, WO 02/02509, WO 02/20466, and WO 03/045306). SNAC, SNAD, 4-CNAB, and the free acid and other salts thereof may be prepared by methods known in the art, such as those described in U.S. Pat. Nos. 5,650,386 and 5,866,536 and International Publication No. WO 02/02509, each of which are hereby incorporated by reference.

Salts of the delivery agent compounds of the present invention may be prepared by methods known in the art. For example, sodium salts may be prepared by dissolving the delivery agent compound in ethanol and adding aqueous sodium hydroxide.

The delivery agent compound may be purified by recrystallization or by fractionation on one or more solid chromatographic supports, alone or linked in tandem. Suitable recrystallization solvent systems include, but are not limited to, acetonitrile, methanol, and tetrahydrofuran. Fractionation may be performed on a suitable chromatographic support such as alumina, using methanol/n-propanol mixtures as the mobile phase; reverse phase chromatography using trifluoroacetic acid/acetonitrile mixtures as the mobile phase; and ion exchange chromatography using water or an appropriate buffer as the mobile phase. When anion exchange chromatography is performed, preferably a 0-500 mM sodium chloride gradient is employed.

The delivery agent may contain a polymer conjugated to it by a linkage group selected from the group consisting of —NHC(O)NH—, —C(O)NH—, —NHC(O), —OOC—, —COO—, —NHC(O)O—, —OC(O)NH—, —CH₂NH—NHCH₂—, —CH₂NHC(O)O—, —OC(O)NHCH₂—, —CH₂NHCOCH₂O—, —OCH₂C(O)NHCH₂—, —NHC(O)CH₂O—, —OCH₂C(O)NH—, —NH—, —O—, and carbon-carbon bond, with the proviso that the polymeric delivery agent is not a polypeptide or polyamino acid. The polymer may be any polymer including, but not limited to, alternating copolymers, block copolymers and random copolymers, which are safe for use in mammals. Preferred polymers include, but are not limited to, polyethylene; polyacrylates; polymethacrylates; poly(oxyethylene); poly(propylene); polypropylene glycol; polyethylene glycol (PEG); and derivatives thereof and combinations thereof. The molecular weight of the polymer typically ranges from about 100 to about 200,000 daltons. The molecular weight of the polymer preferably ranges from about 200 to about 10,000 daltons. In one embodiment, the molecular weight of the polymer ranges from about 200 to about 600 daltons and more preferably ranges from about 300 to about 550 daltons.

Active Agents

Active agents suitable for use in the present invention include biologically active agents and chemically active agents, including, but not limited to, pesticides, pharmacological agents, and therapeutic agents. Suitable active agents include those that are rendered less effective, ineffective or are destroyed in the gastro-intestinal tract by acid hydrolysis, enzymes and the like. Also included as suitable active agents are those macromolecular agents whose physiochemical characteristics, such as, size, structure or charge, prohibit or impede absorption when dosed orally.

For example, biologically or chemically active agents suitable for use in the present invention include, but are not limited to, proteins; polypeptides; peptides; hormones; polysaccharides, and particularly mixtures of muco-polysaccharides; carbohydrates; lipids; small polar organic molecules (i.e. polar organic molecules having a molecular weight of 500 daltons or less); other organic compounds; and particularly compounds which by themselves do not pass (or which pass only a fraction of the administered dose) through the gastro-intestinal mucosa and/or are susceptible to chemical cleavage by acids and enzymes in the gastro-intestinal tract; or any combination thereof.

Further examples include, but are not limited to, the following, including synthetic, natural or recombinant sources thereof: growth hormones, including human growth hormones (hGH), recombinant human growth hormones (rhGH), bovine growth hormones, and porcine growth hormones; growth hormone releasing hormones; growth hormone releasing factor, interferons, including a (e.g., interferon alfacon-1 (available as Infergen® from InterMune, Inc. of Brisbane, Calif.)), β and γ; interleukin-1; interleukin-2; insulin, including porcine, bovine, human, and human recombinant, optionally having counter ions including zinc, sodium, calcium and ammonium; insulin-like growth factor, including IGF-1; heparin, including unfractionated heparin, heparinoids, dermatans, chondroitins, low molecular weight heparin, very low molecular weight heparin and ultra low molecular weight heparin; calcitonin, including salmon, eel, porcine and human; erythropoietin; atrial naturetic factor; antigens; monoclonal antibodies; somatostatin; protease inhibitors; adrenocorticotropin, gonadotropin releasing hormone; oxytocin; leutinizing-hormone-releasing-hormone; follicle stimulating hormone; glucocerebrosidase; thrombopoietin; filgrastim; prostaglandins; cyclosporin; vasopressin; cromolyn sodium (sodium or disodium chromoglycate); vancomycin; desferrioxamine (DFO); bisphosphonates, including alendronate, tiludronate, etidronate, clodronate, pamidronate, olpadronate, and incadronate; parathyroid hormone (PTH), including its fragments; anti-migraine agents such as BIBN-4096BS and other calcitonin gene-related proteins antagonists; glucagon-like peptide 1 (GLP-1); antimicrobials, including antibiotics, anti-bacterials and anti-fungal agents; vitamins; analogs, fragments, mimetics or polyethylene glycol (PEG)-modified derivatives of these compounds; or any combination thereof. Non-limiting examples of antibiotics include gram-positive acting, bacteriocidal, lipopeptidal and cyclic peptidal antibiotics, such as daptomycin and analogs thereof.

According to one embodiment, the active agent is insulin.

According to another embodiment, the active agent is heparin, such as unfractionated heparin or low molecular weight heparin.

The amount of active agent used in a pharmaceutical composition or dosage unit form of the present invention is an amount effective to treat the target indication. However, the amount can be less than that amount when the composition is used in a dosage unit form because the dosage unit form may contain a plurality of delivery agent compound/active agent, such compositions may contain a divided effective amount. The total effective amount can then be administered in cumulative units containing, in total, an effective amount of active agent. Moreover, those skilled in the field will recognize that an effective amount of active agent will vary with many factors including the age and weight of the animal, the animal's physical condition, as well as other factors.

The total amount of active agent to be used of can be determined by methods known to those skilled in the art. However, because the compositions of the invention may deliver active agent more efficiently than compositions containing the active agent without the delivery agent, lower amounts of active agent than those used in prior dosage unit forms or delivery systems can be administered to the subject, while still achieving the same blood levels and/or therapeutic effects.

According to one embodiment, insulin is administered at a dose of about 0.025 to about 1.0 mg per kilogram of body weight of the recipient per day (mg/kg/day), about 0.06 to about 0.25 mg/kg/day, or about 0.09 to about 0.19 mg/kg/day (based on the weight of active agent). The desired dose may be administered either as a single or divided dose.

Generally an effective amount of delivery agent to facilitate the delivery of the active agent is administered with the active agent. According to one embodiment, the amount of delivery agent to active agent on a molar basis ranges from about 100:1 to about 1:1, from about 80:1 to about 2:1, or from about 20:1 to about 10:1. Delivery agent to active agent molar basis ranges may be higher than 100:1 for particular combinations of delivery agents and active agents. Alternatively, delivery agent to active agent ranges may be about 1:1 or lower, such as, e.g., 0.1:1 or lower, with particular combinations of delivery agents and active agents.

Dosage unit forms can also include any one or combination of excipients, disintegrants, lubricants, plasticizers, colorants, flavorants, taste-masking agents, sugars, sweeteners, and salts.

The compositions of the subject invention are useful for administering biologically or chemically active agents to any animals, including but not limited to birds such as chickens, insects, fish, reptiles, mammals (including, but not limited to, rodents, aquatic mammals, domestic animals such as dogs and cats, farm animals such as sheep, pigs, cows and horses, and preferably humans).

Another embodiment of the present invention is a method for the treatment or prevention of a disease or for achieving a desired physiological effect, such as those listed in the table 1 below, in an animal by administering the particles of the present invention. Preferably, an effective amount of the particles for the treatment or prevention of the desired disease or for achieving the desired physiological effect is administered. Specific indications for active agents can be found in the Physicians' Desk Reference (58^{th} Ed., 2004, Medical Economics Company, Inc., Montvale, N.J.), which is herein incorporated by reference. The active agents in the table below include their analogs, fragments, mimetics, and polyethylene glycol-modified derivatives.

TABLE 1

| | Non-Limiting Examples of Disease And |
|---|---|
| Active Agent | Physiological Effect |
| Amylin and Amylin Agonists; | Obesity |
| Adrenocorticotropin; | High Cholesterol (To Lower Cholesterol) |
| Antigens; | Infection |
| Antimicrobials, including Antibiotics, | Infection Including Gram-Positive Bacterial |
| Anti-Bacterials and Anti-Fungal Agents; | Infection |
| non-limiting examples of Antibiotics | |
| include Gram-Positive Acting, | |
| Bacteriocidal, Lipopeptidal and Cyclic | |
| Peptidal Antibiotics, such as Daptomycin | |
| and Analogs thereof; | |
| Anti-Migraine Agents such as BIBN- | Migraines |
| 4096BS and Other Calcitonin Gene- | |
| Related Proteins Antagonists, | |
| Sumatriptan Succinate; | |
| Antivirals including Acyclovir and | Viral Infections |
| Valacyclovir; | |
| Atrial Naturetic Factor; | Vasodilation |
| Argatroban; | Prophylaxis and treatment of thrombosis in |
| | patients with herapin-induced |
| | throbocytopenia (“HIT”), as well as an |
| | anticoagulant therapy in patients who have |
| | or are at risk for HIT undergoing |
| | percutaneous coronary intervention (“PCI”). |
| | Argatroban is also useful to treat thrombotic |
| | and isechemic stroke. |
| Bisphosphonates, including Alendronate, | Osteoporosis; Paget's disease; Inhibits |
| Clodronate, Etidronate, Ibandronate, | osteoclasts and Promotes osteoblastic |
| Incadronate, Minodronate, Neridronate, | activity; treat and/or prevent bone mineral |
| Olpadronate, Pamidronate, Risedronate, | density (bmd) loss; Breast cancer, including |
| Tiludronate, Zoledronate, EB1053, and | as adjuvant therapy for early stage breast |
| YH529; | cancer; Prostate cancer,; Testicular cancer; |
| | Colon cancer; Pancreatic cancer; |
| | Endometrial cancer; Small cell and non- |
| | small cell cancer of the lung; Ovarian |
| | cancer; Cervical cancer; Myeloid leukemia,; |
| | Lymphocyltic leukemia; Lymphoma; |
| | Hepatic tumors; Medullary thyroid |
| | carcinoma; Multiple myeloma; Melanoma |
| | retinoblastoma; Sarcomas of the soft tissue |
| | and bone; Hypercalcemia including |
| | hypercalcemia associated with malignancy; |
| | Osteolytic bone metastases and bone tumors; |
| | prevention of bone complications related to |
| | malignant osteolysis; Osteolytic lesions of |
| | multiple myeloma, fibrous dysplasia; |
| | pediatric osteogenesis imperfecta; |
| | hypercalcemia, urethral (urinary tract) |
| | malignancies, reflex sympathetic dystropy |
| | synodrome, acute back pain after vertebral |
| | crush fracture, chronic inflammatory joint |
| | disease, renal bone disease, extrosseous |
| | calcifications, analgesic, vitamin D |
| | intoxication, periarticular ossifications |
| BIBN4096BS - (1- | Anti-migraine; calcitonin gene-related |
| Piperidinecarboxamide. N-[2-[ [5- | peptide antagonist |
| amino-1-[ [4-(4-pyridinyl)-1- | |
| piperazinyl)carbonyl]pentyl]amino]-1- | |
| [(3,5-dibromo-4-hydroxyphenyl)methyl]- | |
| 2-oxoethyl]-4(1,4-dihydro-2-oxo-3(2H0- | |
| quinazolinyl)-.[R-(R*,S*)]-); | |
| Calcitonin, including Salmon, Eel, | Osteoporosis; Diseases of the bone; bone |
| Porcine and Human; | pain; analgesic (including pain associated |
| | with osteoporosis or cancer) |
| Cholecystokinin (CCK) and CCK | Obesity |
| Agonists including CCK-8; | |
| Cromolyn Sodium (Sodium Or Disodium | Asthma; Allergies |
| Chromoglycate); | |
| CPHPC; | Reduction of amyloid deposits and systemic |
| | amyloidoisis often (but not always) in |
| | connection with Alzheimer's disease, Type II |
| | diabetes, and other amyloid-based diseases |
| Cyclosporine; | Transplant Rejection |
| Desferrioxamine (DFO); | Iron Overload |
| Dipeptidyl peptidase IV (DPP-4) | Diabetes; improving glycemic control (e.g. |
| inhibitors; | treating hypoglycemia), obesity |
| Erythropoietin; | Anemia |
| Exedin and Exedin Agonists, including | Diabetes; Obesity |
| Exendin-3 and Exendin-4; | |
| Filgrastim | Reduce infection in chemotherapy patients |
| Follicle Stimulating Hormone | Regulate reproductive function |
| (recombinant and natural); | |
| Gallium nitrate; | Osteoporosis; Paget's disease; Inhibits |
| | osteoclasts; Promotes osteoblastic activity, |
| | hypercalcemia, including cancer related |
| | hypercalcemia, urethral (urinary tract) |
| | malignancies; anti-tumors, cancers, |
| | including urethral and bladder cancers; |
| | lymphoma; malignancies (including bladder |
| | cancer); leukemia; management of bone |
| | metastases (and associated pain); muliple |
| | myeloma, attenuate immune response, |
| | including allogenic transplant rejections; |
| | disrupt iron metabolism; promote cell |
| | migration; wound repair; to attenuate or treat |
| | infectious processes of mycobacterium |
| | species, including but not limited to |
| | mycobacterium tubercolosis, and |
| | mycobacterium avium complex |
| Glucagon; | Improving glycemic control (e.g. treating |
| | hypoglycemia and controlling hypoglycemic |
| | reactions); obesity; a diagnostic aid in the |
| | radiogical examination of the stomach, |
| | duodenum, small bowel and colon; treat |
| | acute poisoning with cardiovascular agents |
| | including, but not limited to, calcium |
| | channel blockers and beta blockers |
| Glucagon-Like Peptide 1 (GLP-1), | Diabetes; Obesity |
| Glucagon, and Glucagon-Like Peptide 2 | |
| (GLP-2); | |
| Glucocerebrosidase; | Gaucher disease (to metabolize lipoprotein) |
| Gonadotropin Releasing Hormone; | Ovulatory dysfunction (to stimulate |
| | ovulation) |
| Growth Hormone Releasing Factor; | Growth Disorders |
| Growth Hormone Releasing Hormones; | Growth Disorders |
| Growth hormones, including human | Growth Disorders |
| growth hormones (hGH), recombinant | |
| human growth hormones (rhGH), bovine | |
| growth hormones, and porcine growth | |
| hormones; | |
| Heparin, including unfractionated | Thrombosis; prevention of blood coagulation |
| heparin, heparinoids, dermatans, | |
| chondroitins, low molecular weight | |
| heparin, very low molecular weight | |
| heparin ultra low molecular weight | |
| heparin and synthetic heparins including | |
| fondiparinux; | |
| Insulin, including porcine, bovine, | Diabetes; insulin resistance syndrome |
| human, and human recombinant, | |
| optionally having counter ions including | |
| zinc, sodium, calcium and ammonium; | |
| Insulin-Like Growth Factor, including | Diabetes |
| IGF-1; | |
| Interferons, including α (e.g., interferon | Viral infection, including chronic cancer and |
| Alfacon-1 (available as Infergen ® from | multiple sclerosis |
| Intermune, Inc. of Brisbane, Ca)), β, | |
| omega and γ; | |
| Interleukin-1; Interleukin-2; Interleukin- | Viral Infection; Cancer |
| 11; Interleukin-21; | |
| Leutinizing Hormone and Leutinizing | Regulate Reproductive Function |
| Hormone Releasing Hormone; | |
| Leptin (OB Protein); | Obesity |
| Methyphenidate salt; | ADHD, Attention Deficit Disorder, |
| | Dementia, AIDS Dementia Complex, |
| | cognitive decline in HIV-AIDS |
| Monoclonal Antibodies including | To prevent graft rejection; cancer |
| Retuxin, TNF-alpha soluble receptors; | |
| Oxytocin; | Labor dysfunction (to stimulate contractions) |
| Parathyroid Hormone (PTH), including | Osteoporosis; diseases of the bone |
| its fragments, including PTH 1-34 and | |
| PTH 1-38; | |
| Peptide YY (PYY) including PYY | Obesity; Diabetes; Eating Disorders; Insulin |
| Agonists and Fragment 3-36; | Resistance Syndrome |
| Prostaglandins; | Hypertension |
| Protease Inhibitors; | Aids |
| Somatostatin; | Bleeding ulcer; erosive gastritis; variceal |
| | bleeding; diarrhea; acromegaly; TSH- |
| | secreting pituitary adenomas; secretory |
| | pancreatic tumors; carcinoid syndrome; |
| | reduce proptosis/thyroid-associated |
| | ophthalmopathy; reduce macular |
| | edema/retinopathy |
| Thrombopoietin; | Thrombocytopenia |
| Vancomycin; | Treat or prevent antimicrobial-induced |
| | infections including, but not limited to |
| | methacillin-resistant Staphalococcus aureus |
| | and Staph. epidermiditis |
| Vasopressin; | Bed-wetting; antidiuretic |
| Vitamins; and | Vitamin deficiencies |
| Vaccines including those against Anthrax | Prevent and minimize disease |
| or Y. Pestis, Influenza, and Herpes. | |

Controlled or Sustained Release Formulations

The solid dosage forms of the present invention may be formulated so as to prevent or retard break down in the stomach. Controlled release formulations suitable for use in the present invention may, for example, include an enteric coating or may be formulated to erode from the surface.

According to one embodiment, the solid oral dosage forms comprises a therapeutically effective amount of an active agent and a delivery agent, wherein the solid oral dosage form has a disintegration time of about 250 seconds to about 650 seconds when orally administered. In another embodiment, the disintegration time is about 350 to about 550 seconds when orally administered. In one embodiment the disintegration time is greater than 60 seconds when orally administered. In another embodiment, the disintegration time is greater than 400 seconds when orally administered. Disintegration time can be determined in water at 37±2° C. using the method described in USP <701>.

The solid dosage forms of the present invention may be covered by an enteric coating. The enteric coating may serve as the primary control for delaying the release of the drug composition or compositions in the solid dosage form. The enteric coating stays intact in the stomach and prevents or retards release into the stomach in the solid dosage form. Release of the active agent is delayed until the solid dosage form reaches the intestine. Once in the intestine, the higher pH causes release of the active agent. Enteric coatings include, but are not limited to, hydroxypropyl methylcellulose phthalate, hydroxypropyl methylcellulose acetate succinate, polyvinyl acetate phthalate, cellulose acetate trimellitate, cellulose acetate phthalate, poly(methacrylic acid-ethylacrylate), and poly(methacrylic acid-methyl methacrylate). Other enteric coatings which may be used in accordance with the present invention are described in U.S. Pat. No. 5,851,579, which is hereby incorporated by reference.

In one embodiment of the present invention, the enteric coating is applied to the entire tablet, or other dosage form. In one embodiment the enteric coating is applied to a multi-particulate system, such as a system comprising microparticles and/or nanoparticles discussed above.

The solid dosage forms of the present invention may be formulated to erode from the surface of the tablet (or other dosage uniform), or at the surface of the multi-particulate system (e.g. a system comprising microparticles discussed above). These surface erosion formulations slowly dissolve from the surface rather than disintegrate. By controlling the rate of surface erosion, release of the active agent and drug composition of the solid dosage form can be delayed. The surface erosion formulations can be formulated such that substantial release of the active agents or drug compositions do not occur until the solid oral dosage form reaches the intestines.

Enzyme Inhibiting Agents

The solid dosage forms of the present invention (comprising the microparticles or nanoparticles of the present invention and/or having the disintegration times discussed above) may also include enzyme inhibiting agents. Enzyme inhibiting agents incorporated into the solid dosage unit forms may prevent the breakdown of insulin or other active agents that may be sensitive to enzymatic degradation. Enzyme inhibiting agents are described in U.S. Pat. No. 6,458,383 which is hereby incorporated by reference.

Generally, inhibitory agents can be divided into the following classes: inhibitors that are not based on amino acids, including P-aminobenzamidine, FK-448, camostat mesylate and sodium glycocholate; amino acids and modified amino acids, including aminoboronic acid derivatives and n-acetylcysteine; peptides and modified peptides, including bacitracin, phosphinic acid dipeptide derivatives, pepstatin, antipain, leupeptin, chymostatin, elastatin, bestatin, hosphoramindon, puromycin, cytochalasin potatocarboxy peptidase inhibitor, and amastatin; polypeptide protease inhibitors, including aprotinin (bovine pancreatic trypsin inhibitor), Bowman-Birk inhibitor and soybean trypsin inhibitor, chicken egg white trypsin inhibitor, chicken ovoinhibitor, and human pancreatic trypsin inhibitor; complexing agents, including EDTA, EGTA, 1,10-phenanthroline and hydroxychinoline; and mucoadhesive polymers and polymer-inhibitor conjugates, including polyacrylate derivatives, chitosan, cellulosics, chitosan-EDTA, chitosan-EDTA-antipain, polyacrylic acid-bacitracin, carboxymethyl cellulose-pepstatin, polyacrylic acid-Bowman-Birk inhibitor.

The choice and levels of the enzyme inhibitor are based on toxicity, specificity of the proteases and the potency of inhibition, and will be apparent to those skilled in the art.

Without wishing to be bound by theory, it is believed that an inhibitor can function solely or in combination as: a competitive inhibitor, by binding at the substrate binding site of the enzyme, thereby preventing the access to the substrate (examples of inhibitors believed to operate by this mechanism are antipain, elastatinal and the Bowman Birk inhibitor); a non-competitive inhibitor that can be simultaneously bound to the enzyme site along with the substrate, as their binding sites are not identical; and/or a complexing agent due to loss in enzymatic activity caused by deprivation of essential metal ions out of the enzyme structure.

Solid Pharmaceutical Composition Embodiment

This application hereby incorporates by reference International Publication No. WO 2005/004900 and its priority document U.S. Provisional Application No. 60/486,495, filed Jul. 11, 2003, in their entireties.

The pharmacologically active agents suitable for use in the solid pharmaceutical composition of the instant invention include both therapeutic as well as preventative agents and is directed particularly to agents which by themselves do not pass or which pass only a small amount of the administered dose through the gastro-intestinal mucosa and/or are susceptible to cleavage by acids and enzymes in the gastro-intestinal tract. The pharmacologically active agents include, but are not limited to proteins; polypeptides; hormones; polysaccharides including mixtures of muco-polysaccharides; carbohydrates; lipids; and combinations thereof.

Specific examples of pharmacologically active agents include, but are not limited to, the following, including synthetic, natural or recombinant sources thereof: growth hormone, including human growth hormones (hGH), recombinant human growth hormones (rhGH), bovine growth hormones, and porcine growth hormones; growth hormone-releasing hormones; interferons, including α, β, and γ-interferon; interleukin-1; interleukin-2; insulin, including porcine, bovine, human, and human recombinant, optionally having counter ions including sodium, zinc, calcium and ammonium; insulin-like growth factor, including IGF-1; heparin, including unfractionated heparin, heparinoids, dermatans, chondroitins, low, very low and ultra low molecular weight heparin; calcitonin, including salmon, porcine, eel, chicken and human; erythopoietein; atrial naturetic factor; antigens; monoclonal antibodies; somatostatin; protease inhibitors; adrenocorticotropin, gonadotropin releasing hormone; oxytocin; leutinizing-hormone-releasing hormone; follicle stimulating hormone; glucocerebrosidase; thrombopoietin; filgrastim; prostaglandins; cyclosporin; vasopressin; cromolyn sodium (sodium or disodium chromoglycate); vancomycin; desferrioxamine(DFO); parathyroid hormone (PTH), including its fragments; antimicrobials, including anti-fungal agents; vitamins; analogs, fragments, mimetics or polyethylene glycol(PEG)-modified derivatives of these compounds; or any combination thereof.

An interesting pharmacologically active agent is a pharmacologically active peptide, particularly bone active agents, and even more particularly calcitonin.

Bone active agents include classes of agents which display in vivo pharmacological activity in animals such as stabilization, healing, or growth of bone, deceleration or inhibition of bone turnover, deceleration or inhibition of bone resorption, inhibition of osteoclast activity, and stimulation of osteoblast activity. Some of these agents may be peptidic, for example calcitonins, parathyroid hormone (PTH), PTH fragments, analogs and releasers, and Transforming Growth Factors(TGFs) fragments, analogs and releasers. The bone active agents may also be small molecule non-peptidic structures which show in vivo pharmacological bone activities as described above in this paragraph.

A known class of such pharmacologically active agents, calcitonins, have varying pharmaceutical utility and are commonly employed in the treatment of e.g. Paget's disease, hypercalcemia and postmenopausal osteoporosis. Various calcitonins, including salmon, pig and eel calcitonin are commercially available and commonly employed for the treatment of e.g. Paget's disease, hypercalcemia of malignancy and osteoporosis. The calcitonin can be any calcitonin, including natural, synthetic or recombinant sources thereof, as well as calcitonin derivatives such as 1,7-Asu-eel calcitonin. The compositions can comprise a single calcitonin or any combination of two or more calcitonins. The preferred calcitonin is synthetic salmon calcitonin.

The calcitonins are commercially available or may be synthesized by known methods.

The amount of pharmacologically active agent is generally an amount effective to accomplish the intended purpose, e.g. a therapeutically effective amount. However, the amount can be less than that amount when a plurality of the compositions are to be administered, i.e., the total effective amount can be administered in cumulative dosage units. The amount of active agent can also be more than the effective amount when the composition provides sustained release of the pharmacologically active agent. The total amount of active agent to be used can be determined by methods known to those skilled in the art. However, because the compositions may deliver the active agent more efficiently than prior compositions, less amounts of active agent than those used in prior dosage unit forms or delivery systems can be administered to a subject while still achieving the same blood levels and/or therapeutic effects.

When the pharmacologically active agent is salmon calcitonin, the appropriate dosage will, of course, vary depending upon, for example, the host and the nature and severity of the condition being treated. However, in general, satisfactory results will be obtained systemically at daily dosages of from about 0.5 g/kg to about 10 g/kg animal body weight, preferably 1 g/kg to about 6 body weight.

The pharmacologically active agent generally comprises from 0.05 to 70 percent by weight relative to the total weight of the overall pharmaceutical composition, preferably an amount of from 0.01 to 50 percent by weight, more preferably 0.3 to 30 percent by weight relative to the total weight of the overall pharmaceutical composition.

The pharmaceutically acceptable inactive excipients may include polymers and inactive compounds which for example, aid the formulation or manufacturing of the solid oral dosage form contemplated by the present invention or which may aid the release of the solid oral composition in the gastro-intestinal environment.

The pharmaceutical inactive ingredients, referred to above, for example optionally include crospovidones and povidones, which may be any crospovidone and povidone. Crospovidone is a synthetic crosslinked homopolymer of N-vinyl-2-pyrrolidone, also called 1-ethenyl-2-pyrrolidinone, having a molecular weight of 1,000,000 or more. Commercially available crospovidones include Polyplasdone XL, PolyplasdoneXL-10, Polyplasdone INF-10 available from ISP, Kollidon CL, available from BASF Corporation. The preferred crospovidone is Polyplasdone XL.

Povidone is a synthetic polymer consisting of linear 1-vinyl-2-pyrrolidinone groups having a molecular weight generally between 2,500 and 3,000,000. Commercially available povidones include Kollidon K-30, Kollidon K-90F available from BASF Corporation and Plasdone K-30 and Plasdone K-29/32, available from ISP.

As mentioned above, the crospovidones and povidones are commercially available. Alternatively, they may be synthesized by known processes.

The crospovidone, povidone or combination thereof is generally present in the compositions in an amount of from 0.5 to 50 percent by weight relative to the total weight of the overall pharmaceutical composition, preferably an amount of from 2 to 25 percent, more preferably 5 to 20 percent by weight relative to the total weight of the pharmaceutical composition.

The delivery agents useful in the solid pharmaceutical composition are any agents useful for delivering the particular pharmacologically active agent. Suitable delivery agents are any one of the 123 modified amino acids disclosed in U.S. Pat. No. 5,866,536 or any one of the 193 modified amino acids described in the U.S. Pat. No. 5,773,647 or any combination thereof. The contents of the aforementioned U.S. Pat. Nos. 5,773,647 and 5,866,536 are hereby incorporated by reference in their entirety. In addition, the delivery agent can be the disodium salt of any of the aforementioned modified amino acids as well as ethanol solvates and hydrates thereof. Suitable compounds include compounds of the following formula I 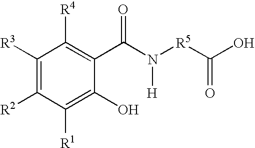
wherein:

R¹, R², R³, and R⁴ are independently hydrogen, —OH, —NR⁶R⁷, halogen, C₁-C₄ alkyl, or C₁-C₄ alkoxy;

R⁵ is a substituted or unsubstituted C₂-C₆ alkylene, substituted or unsubstituted C₂-C₁₆ alkenylene, substituted or unsubstituted C₁-C₁₂ alkyl(arylene), or substituted or unsubstituted aryl(C₁-C₁₂ alkylene); and

R₆ and R⁷ are independently hydrogen, oxygen, or C₁-C₄ alkyl; and hydrates and alcohol solvates thereof. The compounds of formula I as well as their disodium salts and alcohol solvates and hydrates thereof are described in WO 00/059863, along with methods for preparing them.

The disodium salt may be prepared from the ethanol solvate by evaporating or drying the ethanol solvate by methods known in the art to form the anhydrous disodium salt. Drying is generally carried out at a temperature of from about 80 to about 120° C., preferably from about 85 to about 90° C., and most preferably at about 85° C. The drying step is generally performed at a pressure of 26″ Hg or greater. The anhydrous disodium salt generally contains less than about 5% by weight of ethanol and preferably less than about 2% by weight of ethanol, based on 100% total weight of anhydrous disodium salt.

The disodium salt of the delivery agent can also be prepared by making a slurry of the delivery agent in water and adding two molar equivalents of aqueous sodium hydroxide, sodium alkoxide or the like. Suitable sodium alkoxide include, but are not limited to, sodium methoxide, sodium ethoxide, and combinations thereof.

A still further method of preparing the disodium salt is by reacting the delivery agent with one molar equivalent of sodium hydroxide to yield the disodium salt.

The disodium salt can be isolated as a solid by concentrating the solution containing the disodium salt to a thick paste by vacuum distillation. This paste may be dried in a vacuum oven to obtain the disodium salt of the delivery agent as a solid. The solid can also be isolated by spray drying an aqueous solution of the disodium salt.

The delivery agents may be prepared by methods known in the art, e.g., as mentioned above, by methods described in U.S. Pat. Nos. 5,773,647 and 5,866,536.

The ethanol solvates, as described in the aforementioned International Publication No. WO 00/059863, include, but are not limited to, a molecular or ionic complex of molecules or ions of ethanol solvent with molecules or ions of the disodium salt of the delivery agent. Typically, the ethanol solvate contains about one ethanol molecule or ion for every molecule of disodium salt of the delivery agent.

The ethanol solvate of the disodium salt of the delivery agent can be prepared by dissolving the delivery agent in ethanol. Typically, each gram of delivery agent is dissolved in from about 1 to about 50 mL of ethanol and generally, from about 2 to about 10 mL of ethanol. The delivery agent/ethanol solution is then reacted with a molar excess of a sodium containing salt, such as a monosodium containing salt, relative to delivery agent, i.e. for every mole of delivery agent there is more than one mole of sodium cations, yielding the ethanol solvate. Suitable monosodium salts include, but are not limited to, sodium hydroxide; sodium alkoxides, such as sodium methoxide and sodium ethoxide; and any combination of the foregoing. Preferably, at least about two molar equivalents of the monosodium containing salt are added to the ethanol solution, i.e. for every mole of delivery agent there is at least about two moles of sodium cations. Generally, the reaction is performed at or below the reflux temperature of the mixture, such as at ambient temperature. The ethanol solvate is then recovered by methods known is the art, such as, concentration of the resulting slurry at atmospheric distillation, cooling the concentrated slurry and filtering the solid. The recovered solid can then be vacuum dried to obtain the ethanol solvate.

The hydrates of the disodium salts of the delivery agents may be prepared by drying the ethanol solvate to form an anhydrous disodium salt, as described above, and hydrating the anhydrous disodium salt. Preferably, the monohydrate of the disodium salt is formed. Since the anhydrous disodium salt is very hydroscopic, the hydrate forms upon exposure to atmospheric moisture. Generally, the hydrating step is performed at from about ambient temperature to about 50° C., preferably ambient temperature to about 30° C. and in an environment having at least 50% relative humidity. Alternatively, the anhydrous disodium salt may be hydrated with steam.

The preferred delivery agents for the solid pharmaceutical composition are N-(5-chlorosalicyloyl)-8-aminocaprylic acid (5-CNAC acid), N-(10-[2-hydroxybenzoyl]-amino) decanoic acid (SNAD acid), N-(8-[2-hydroxybenzoyl]amino) caprylic acid (SNAC acid) and their monosodium and disodium salts, ethanol solvates of their sodium salts and the monohydrates of their sodium salts and any combinations thereof. The most preferred delivery agent is the disodium salt of 5-CNAC acid and the monohydrate thereof.

The delivery agents 5 CNAC acid, SNAD acid, and SNAC acid (and their salts) are very water soluble and nearly fully, i.e. greater than 90%, absorbed by the gastro-intestinal tract whether it is ingested in micronized or coarse form. However, when a micronized form of one of these carrier agents is employed in the composition, the absorption of the pharmacologically active agent of the present composition is more completely absorbed into the blood stream.

A micronized form of the delivery agent, which is utilized in preparation of the solid pharmaceutical composition or solid oral dosage form of the present invention, is defined as a delivery agent which, when added to the present composition mixture of pharmacologically active agent and pharmaceutical inactive ingredients, has an average particle size of less than 40 micrometers. Desirably the delivery agent of the present invention has a micronized form which is defined as an average particle size of less than 20 microns. More interestingly, the delivery agent for the present invention has a micronized form which is defined as an average particle size of less than 10 microns.

Micronized forms of the delivery agent of the present invention may be prepared by grinding it in a grinding mill which is acceptable for grinding pharmaceutical ingredients and which is capable of grinding the pharmaceutical ingredients and/or delivery agent to a fine and uniform micronized particle size. An example of such a grinding mill is an Air Jet Mill GemT &commat; (Copley Scientific, Ltd., Nottingham, UK). The finely ground delivery agent either separately or finely ground delivery agent plus any combination of finely ground additional ingredients of the present invention may then be screened, for example, over a mesh screen having the appropriate openings, in order to allow only those ingredients which have the required particle size to pass through and be collected for use in the present invention.

The solid pharmaceutical compositions typically contain a delivery effective amount of one or more of the delivery agents, i.e. an amount sufficient to deliver the active agent for the desired effect. Generally, the delivery agent is present in an amount of 2.5% to 99.4% by weight, more preferably 25% to 50% by weight.

The solid pharmaceutical compositions may be provided as a capsule including a soft-gel capsule, tablet, caplet or other solid oral dosage form, all of which can be prepared by methods well known in the art.

The solid pharmaceutical compositions may additionally comprise additives in amounts customarily employed including, but not limited to, a pH adjuster, a preservative, a flavorant, a taste-masking agent, a fragrance, a humectant, a tonicifier, a colorant, a surfactant, a plasticizer, a lubricant such as magnesium stearate, a flow aid, a compression aid, a solubilizer, an excipient, a diluent such as microcrystalline cellulose, e. g. Avicel PH 102® (supplied by FMC Corporation, 1735 Market Street Philadelphia, Pa. 19103, USA), or any combination thereof. Other additives may include phosphate buffer salts, citric acid, glycols, and other dispersing agents.

The solid pharmaceutical composition may also include one or more enzyme inhibitors, such as actinonin or epiactinonin and derivatives thereof; aprotinin, Trasylol and Bowman-Birk inhibitor.

Further, a transport inhibitor, i.e. a ρ-glycoprotein such as Ketoprofin, may be present in the compositions of the present invention.

Preferably, the solid pharmaceutical compositions include a diluent, such as Avicel®, and a lubricant, such as magnesium stearate.

The solid pharmaceutical compositions can be prepared by first grinding either the delivery agent or the delivery agent with any combination of the additional ingredients of the present composition to a micronized particle size. The micronized delivery agent or micronized delivery agent plus micronized additional ingredients of the present invention may then be further processed by conventional methods e.g. by blending a mixture of the active agent or active agents, the delivery agent, the crospovidone or povidone and other ingredients, kneading, and filling into capsules or, instead of filling into capsules, molding followed by further tableting or compression-molding to give tablets. In addition, a solid dispersion may be formed by known methods followed by further processing to form a tablet or capsule.

Preferably, the ingredients in the solid pharmaceutical compositions are homogeneously or uniformly mixed throughout the solid dosage form.

The solid pharmaceutical compositions may be administered to deliver an active agent to any animal in need thereof, including, but not limited to, mammals, such as rodents, cows, pigs, dogs, cats, and primates, particularly humans.

### EXAMPLES

The following examples illustrate the invention without limitation. All parts are given by weight unless otherwise indicated.

### Example 1

1. Test Articles

a. Co-Processed Insulin/Delivery Agent Microparticles Used for Site Specific, In Situ Experiment and Oral Gavage Experiments

Recombinant human zinc insulin (50 mg) and sodium 4-CNAB (7.5 g) were dissolved in 50 ml of deionized water. The clear solution was dried with nitrogen flow at room temperature for 24 hours. The obtained coprocessed cake was milled into fine particles, which were then sieved through a 40/60 mesh screen to obtain microparticles of a specific size range. The size of the microparticles used in the current study ranged from 250 to 420 μm. These microparticles contained by weight 0.55% of insulin, 9.5% of water and 89.5% of delivery agent. A total of approximately 90% (w/w) of insulin was recovered from this process.

Particles were measured by passing them through seives with different size openings (850 μm, 425 μm, 250 μm, 150 μm, 45 μm). With this method, it can be determined that the median particle size ranges from about 45 to about 850 μm, from about 45 to about 150 μm, from about 150 to about 250 μm, from about 250 to about 425 μm, or from about 425 to about 850 μm.

Insulin content in the microparticles was measured with reversed phase HPLC (Phenomenex column: Luna 3u C18 (2) 100 Å, 150×4.6 mm, 3 micro; mobile phases: A, 0.1% TFA in water; B, 0.1% TFA in acetonitrile; Detector: UV280 nm). Water contents of the particles were measured with a 737 KF coulometer.

b. Capsules Loaded with the Microparticles for Oral Gavage

Gelatin capsules (size #9) were used in the rat studies. The necessary amount of microparticles loaded manually into the gelatin capsules were determined based on an average rat body weight of 350 mg. Each loaded capsule contained approximately 16 mg of the microparticles (equivalent to 0.0875 mg of insulin).

c. Insulin/Delivery Agent Mini-Tablets for Oral Gavage Experiment

Insulin was well mixed with delivery agent at a ratio of 1:150 (w/w), which corresponded to 0.67% (w/w) of insulin. Based on an average rat body weight of 350 mg, a total amount of 26.43 mg of the mixed powder, which contained 0.175 mg of insulin and 26.26 mg of delivery agent, was directly compressed into tablets under a pressure of 1000 psi in a Carver press. The cylindrical mini-tablets were 2 mm in diameter and 6 mm in height.

d. Capsules Loaded with Insulin/Delivery Agent Physical Blend for Oral Gavage Experiment

Insulin was well mixed with delivery agent at a ratio of 1:150 (w/w). The amount of insulin and delivery agent mixture loaded manually into the gelatin capsules (size #9) were determined based on an average rat body weight of 350 mg. Each capsule contained 26.43 mg of the mixture (equivalent to 0.175 mg insulin).

2. Direct Dosing Procedures for In Situ Experiments

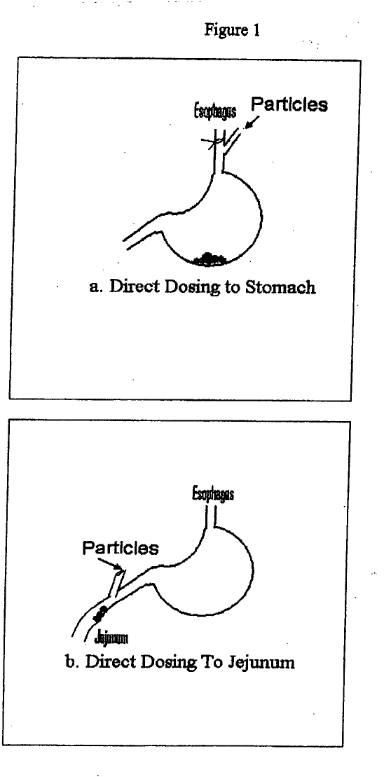

A schematic of the direct dosing procedure is shown in FIG. 1. Surgery was carried out in a clean environment using a clean lab coat, mask, safety goggle, gloves and surgical cap. Anesthesia was induced to the Sprague Dawley rats with 5% isoflurane as an induction concentration, and maintained at 2% isoflurane in pure oxygen to the completion of the study.

a. Stomach Direct Dosing

After the right jugular vein was catheterized for sampling blood, the skin over the esophagus and trachea was dissected, and the musculus digastricul venter rostralis (protective muscular bundles) was identified and dissected to make an access toward the esophagus. The esophagus was partially severed, and inserted with a 12 cm PE204 tubing for a segment of the esophagus measuring 6-9 cm. The dosing formulation was introduced through this tubing using a blunt wire to push in the microparticles. After dosing, the esophagus was ligated with a 3-0 silk suture for preventing any leakage from the stomach.

b. Jejunum Direct Dosing

After the right jugular catheterization for the blood sampling, the abdominal cavity was opened by dissecting the linea alba toward the sternum, thus exposing the xiphoid cartilage. The most proximal segment of jejunum was first identified. A less vascularized section of the proximal jejunum was partially nipped, and a dosing tube was introduced toward the distal end. After dosing, the dosing tube was removed, and a 2 cm PE206 tubing was pushed in, and placed so that the nipped wound was located in the middle of both ends of the 2 cm tubing. A suture was tied around the tubing with jejunum at both ends, and the wound was closed with a drop of a vetbond™ tissue adhesive (available from 3M of St. Paul, Minn.).

3. Oral Gavage Procedures

Studies were carried out in Sprague Dawley rats (body weight was approximately 350 grams) by oral gavage administration. The mini tablets or capsules were administrated orally in rats using a modified gavage tubing with a trocar. Rats were fasted for about 24 hours and anesthetized by intramuscular administration of ketamine (44 mg/kg) and thorazine (1.5 mg/kg). At pre-determined time intervals, blood samples were drawn from the tail artery and were appropriately prepared as either plasma or serum for glucose and insulin bioassays. The animal was sacrificed at the end of the experiment and rat gastrointestinal mucosa was observed for any sign of local toxicity.

4. Bioassay Procedures

Rat serum concentrations of insulin were determined using Insulin ELISA Test Kit (DSL Inc.). The limit of quantitation (LOQ) has been established at 12.5 μU/mL, with the calibrated linear range of the assay up to 250 μU/mL. Changes in blood glucose levels were measured using a glucometer.

5. Results

a. Site Specific Study (In Situ) Results

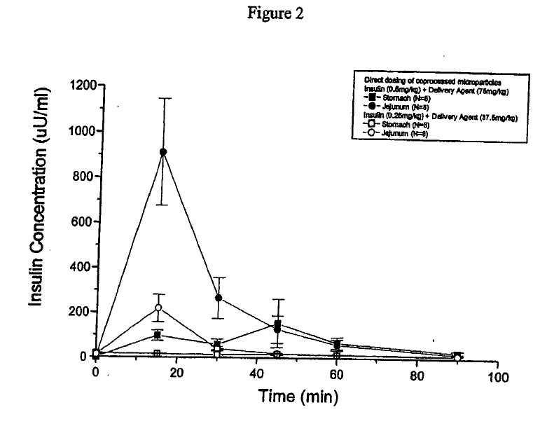

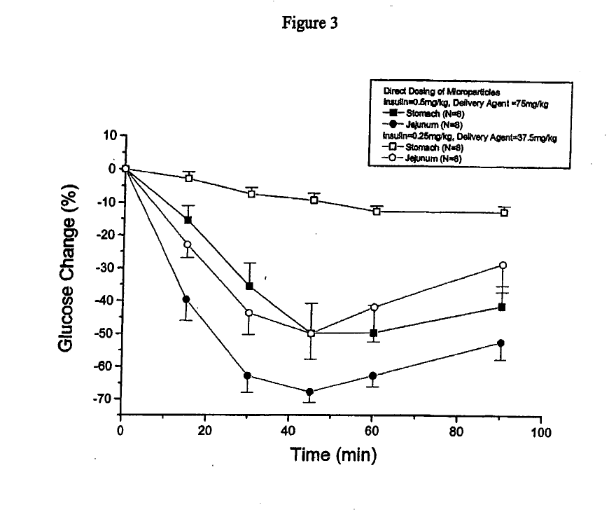

The concentration of insulin and the change in glucose level following direct dosing of the coprocessed microparticles to the stomach and the jejunum are shown in FIGS. 2 and 3, respectively. The individual data are listed in Tables 2 to 5.

Insulin concentration from dosing to the jejunum reached a maximum value at the first sampling point (tₘₐₓ≦15 min) from each formulation. The corresponding tₘᵢₙ of glucose occurred approximately 30 min. later.

TABLE 2
Direct dosing of coprocessed microparticles to the stomach
*Insulin (0.5 mg/kg), Delivery Agent (75 mg/kg))
1) Insulin

| Insulin | Stomach | | | | | | | | | | | |
|---|---|---|---|---|---|---|---|---|---|---|---|---|
| Time | Rat | | | | | | | | | | | |
| (min) | #1 | #2 | #3 | #4 | #5 | #6 | #7 | #8 | mean | SD | SEM | CV |
| 0 | 12.5 | 12.5 | 12.5 | 12.5 | 12.5 | 12.5 | 12.5 | 12.9 | 0.0 | 0.0 | 0.0 | |
| 15 | 114.5 | 72.8 | 80.9 | 12.6 | 210.0 | 12.5 | 118.7 | 158.5 | 97.6 | 68.1 | 24.1 | 69.8% |
| 30 | 95.3 | 19.3 | 35.5 | 12.5 | 211.0 | 12.5 | 15.1 | 66.0 | 58.3 | 68.4 | 24.2 | 117.2% |
| 45 | 62.8 | 12.5 | 894.0 | 12.5 | 213.0 | 12.5 | 15.0 | 12.5 | 154.3 | 306.7 | 108.5 | 198.8% |
| 60 | 18.2 | 12.5 | 157.0 | 12.5 | 174.0 | 140.3 | 12.5 | 12.5 | 67.4 | 74.7 | 26.4 | 110.9% |
| 90 | 12.5 | 12.5 | 12.5 | 12.5 | 61.3 | 12.5 | 12.5 | 74.2 | 26.3 | 25.8 | 9.1 | 98.1% |
| AUC_{0→90} | 4780 | 2132 | 18970 | 1127 | 14438 | 4001 | 2795 | 5046 | 6661 | 6451 | 2281 | 96.8% |

2) Glucose

| Change | | | | | | | | | | | | |
|---|---|---|---|---|---|---|---|---|---|---|---|---|
| from | | | | | | | | | | | | |
| base | | | | | | | | | | | | |
| line | Stomach | | | | | | | | | | | |
| Time | Rat | | | | | | | | | | | |
| (min) | #1 | #2 | #3 | #4 | #5 | #6 | #7 | #8 | Mean | SD | SEM | CV |
| 0 | 0 | 0 | 0 | 0 | 0 | 0 | 0 | 0 | 0 | 0 | 0 | |
| 15 | −9.9 | −21.0 | −12.6 | −38.1 | −6.6 | −24.8 | 3.0 | −13.9 | −15.5 | 12.5 | 4.4 | −80.6% |
| 30 | −44.3 | −43.8 | −25.8 | −56.1 | −9.8 | −54.4 | −3.4 | −47.6 | −35.7 | 20.2 | 7.1 | −56.6% |
| 45 | −75.0 | −50.9 | −30.2 | −73.6 | −17.1 | −73.6 | −14.8 | −64.2 | −49.9 | 25.8 | 9.1 | −51.6% |
| 60 | −80.7 | −51.3 | −31.1 | −66.1 | −18.7 | −72.8 | −21.9 | −55.1 | −49.7 | 23.5 | 8.3 | −47.3% |
| 90 | −62.3 | −32.1 | −35.2 | −59.7 | −20.3 | −63.6 | −29.5 | −28.3 | −41.4 | 17.5 | 6.1 | −42.3% |

(two rat data were removed, rat #1-2 stomach)

TABLE 3
Direct dosing of coprocessed microparticles to the jejunum data
(Insulin (0.5 mg/kg), Delivery Agent (75 mg/kg))
1) Insulin

| Insulin | | | | | | | | | | | | |
|---|---|---|---|---|---|---|---|---|---|---|---|---|
| Time | Jejunum | | | | | | | | | | | |
| (min) | #9 | #10 | #11 | #12 | #13 | #14 | #15 | #16 | mean | SD | SEM | CV |
| 0 | 12.5 | 12.5 | 12.5 | 12.5 | 12.5 | 12.5 | 12.5 | 12.5 | 0.0 | 0.0 | 0.0 | |
| 15 | 413.2 | 1193.3 | 669.4 | 1177.5 | 2270.6 | 228.9 | 954.4 | 374.9 | 910.3 | 661.1 | 233.8 | 72.6% |
| 30 | 354.3 | 148.4 | 70.5 | 64.7 | 481.0 | 168.4 | 782.9 | 57.4 | 265.9 | 258.2 | 91.3 | 97.1% |
| 45 | 79.5 | 28.0 | 20.5 | 26.5 | 170.8 | 148.6 | 531.6 | 12.5 | 127.3 | 174.3 | 61.6 | 137.0% |
| 60 | 23.1 | 14.7 | 12.5 | 16.8 | 71.6 | 117.0 | 200.1 | 12.5 | 58.5 | 68.4 | 24.2 | 116.9% |
| 90 | 12.5 | 12.5 | 12.5 | 12.5 | 30.8 | 12.5 | 37.5 | 12.5 | 17.9 | 10.2 | 3.6 | 56.8% |
| AUC_{0→90} | 13506 | 21158 | 11969 | 19690 | 46003 | 11102 | 39192 | 7235 | 21232 | 14054 | 4969 | 66.2% |

2) Glucose

| Change | | | | | | | | | | | | |
|---|---|---|---|---|---|---|---|---|---|---|---|---|
| from | | | | | | | | | | | | |
| base | | | | | | | | | | | | |
| line | | | | | | | | | | | | |
| Time | Jejunum | | | | | | | | | | | |
| (min) | #9 | #10 | #11 | #12 | #13 | #14 | #15 | #16 | Mean | SD | SEM | CV |
| 0 | 0 | 0 | 0 | 0 | 0 | 0 | 0 | 0 | 0 | 0 | 0 | |
| 15 | −50.8 | −35.6 | −16.7 | −16.0 | −61.1 | −38.2 | −62.8 | −36.3 | −39.7 | 17.9 | 6.3 | −45.0% |
| 30 | −67.3 | −65.5 | −59.5 | −35.8 | −81.8 | −62.5 | −78.1 | −52.9 | −62.9 | 14.4 | 5.1 | −22.9% |
| 45 | −64.4 | −68.5 | −76.3 | −56.4 | −74.4 | −71.6 | −78.1 | −53.3 | −67.9 | 9.2 | 3.2 | −13.5% |
| 60 | −62.7 | −62.5 | −71.3 | −62.7 | −62.1 | −65.6 | −74.0 | −42.2 | −62.9 | 9.5 | 3.4 | −15.1% |
| 90 | −62.4 | −49.8 | −69.7 | −55.6 | −41.8 | −44.2 | −69.4 | −26.7 | −52.5 | 14.9 | 5.3 | −28.3% |

(two rat data were removed, rat# 2 jejunum and rat# 4 jejunum)

TABLE 4
Direct dosing of coprocessed microparticles to the stomach
(Insulin (0.25 mg/kg), Delivery Agent (37.5 mg/kg))
1) Insulin

| Time | stomach | | | | | | | | | | | |
|---|---|---|---|---|---|---|---|---|---|---|---|---|
| (min) | sto-1 | sto-2 | sto-3 | sto-4 | sto-5 | sto-6 | sto-7 | sto-8 | mean | SD | SEM | CV |
| 0 | 12.5 | 12.5 | 41.2 | 21.5 | 12.5 | 12.5 | 12.5 | 12.5 | 17.2 | 9.5 | 3.4 | 55.4% |
| 15 | 12.5 | 20.8 | 14.7 | 12.5 | 12.5 | 12.5 | 26.4 | 12.5 | 16.0 | 4.9 | 1.7 | 30.7% |
| 30 | 12.5 | 12.5 | 12.5 | 12.5 | 12.5 | 12.5 | 12.5 | 12.5 | 12.5 | 0.0 | 0.0 | 0.0% |
| 45 | 48.8 | 12.5 | 12.5 | 12.5 | 12.5 | 12.5 | 12.5 | 12.5 | 17.0 | 12.0 | 4.3 | 70.4% |
| 60 | 12.5 | 12.5 | 12.5 | 12.5 | 12.5 | 12.5 | 12.5 | 12.5 | 12.5 | 0.0 | 0.0 | 0.0% |
| 90 | 12.5 | 12.5 | 12.5 | 12.5 | 12.5 | 12.5 | 12.5 | 12.5 | 12.5 | 0.0 | 0.0 | 0.0% |
| AUC_{0→90} | 1670 | 1250 | 1373 | 1193 | 1125 | 1125 | 1334 | 1125 | 1274 | 187 | 66.0 | 14.6& |

2) Glucose

| Change | | | | | | | | | | | | |
|---|---|---|---|---|---|---|---|---|---|---|---|---|
| from | | | | | | | | | | | | |
| base | | | | | | | | | | | | |
| line | Stomach | | | | | | | | | | | |
| Time | Rat | | | | | | | | | | | |
| (min) | #1 | #2 | #3 | #4 | #5 | #6 | #7 | #8 | mean | SD | SEM | CV |
| 0 | 0 | 0 | 0 | 0 | 0 | 0 | 0 | 0 | 0 | 0 | 0.0 | |
| 15 | −2.2 | −12.3 | 2.2 | 0.8 | −2.2 | −12.3 | 2.2 | 0.8 | −2.9 | 5.7 | 2.0 | −196.5% |
| 30 | −14.4 | −10.1 | −6.5 | 0.8 | −14.4 | −10.1 | −6.5 | 0.8 | −7.6 | 5.6 | 1.9 | −73.7% |
| 45 | −15.8 | −8.8 | −12.7 | 0.0 | −15.8 | −8.8 | −12.7 | 0.0 | −9.3 | 5.9 | 2.1 | −63.4% |
| 60 | −15.8 | −11.4 | −17.9 | −5.8 | −15.8 | −11.4 | −17.9 | −5.8 | −12.7 | 4.6 | 1.6 | −36.2% |
| 90 | −19.1 | −16.3 | −8.6 | −6.6 | −19.1 | −16.3 | −8.6 | −6.6 | −12.7 | 5.2 | 1.8 | −40.9% |

TABLE 5
Direct dosing of coprocessed microparticles to the jejunum data
(Insulin (0.25 mg/kg), Delivery Agent (37.5 mg/kg))
1) Insulin

| Time | jejunum | | | | | | | | | | | |
|---|---|---|---|---|---|---|---|---|---|---|---|---|
| (min) | jej-1 | jej-2 | jej-3 | jej-4 | jej-5 | jej-6 | jej-7 | jej-8 | mean | SD | SEM | CV |
| 0 | 12.5 | 12.5 | 12.5 | 12.5 | 12.5 | 12.5 | 12.5 | 12.5 | 12.5 | 0.0 | 0.0 | 0.0% |
| 15 | 428.5 | 532.4 | 232.6 | 12.5 | 62.0 | 186.6 | 79.6 | 160.5 | 219.2 | 170.7 | 60.4 | 77.8% |
| 30 | 67.9 | 100.8 | 44.7 | 12.5 | 15.5 | 12.5 | 14.2 | 49.3 | 39.7 | 30.4 | 10.7 | 76.4% |
| 45 | 16.8 | 40.8 | 26.6 | 12.5 | 12.5 | 12.5 | 12.5 | 12.5 | 18.4 | 9.7 | 3.4 | 52.6% |
| 60 | 12.5 | 24.7 | 17.3 | 12.5 | 12.5 | 12.5 | 12.5 | 12.5 | 14.6 | 4.1 | 1.5 | 28.1% |
| 90 | 12.5 | 12.5 | 12.5 | 12.5 | 12.5 | 12.5 | 12.5 | 12.5 | 12.5 | 0.0 | 0.0 | 0.0% |
| AUG_{0→90} | 8261 | 10947 | 5229 | 1125 | 1913 | 3737 | 2157 | 3897 | 4658 | 3392 | 1199 | 72.8% |

2) Glucose

| Change | | | | | | | | | | | | |
|---|---|---|---|---|---|---|---|---|---|---|---|---|
| from | | | | | | | | | | | | |
| base | | | | | | | | | | | | |
| line | | | | | | | | | | | | |
| Time | Jejunum | | | | | | | | | | | |
| (min) | #9 | #10 | #11 | #12 | #13 | #14 | #15 | #16 | Mean | SD | SEM | CV |
| 0 | 0 | 0 | 0 | 0 | 0 | 0 | 0 | 0 | 0 | 0 | 0.0 | |
| 15 | −29.7 | −20.5 | −35.8 | 1.3 | −28.9 | −18.2 | −25.9 | −25.6 | −22.9 | 11.2 | 3.9 | −48.9% |
| 30 | −52.2 | −54.5 | −41.9 | −1.3 | −50.0 | −60.3 | −36.8 | −52.8 | −43.7 | 18.7 | 6.5 | −42.8% |
| 45 | −63.6 | −65.3 | −43.8 | −0.7 | −56.8 | −69.2 | −40.8 | −59.7 | −59.0 | 22.3 | 7.8 | −37.8% |
| 60 | −56.9 | −69.4 | −33.8 | 11.6 | −56.8 | −62.1 | −27.2 | −55.7 | −41.9 | 28.1 | 10.6 | −67.1% |
| 90 | −63.9 | −59.7 | −26.9 | 8.3 | −50.0 | −31.8 | −11.8 | −28.4 | −28.6 | 22.7 | 8.5 | −79.4% |

b. Results from Oral Gavage Experiments Using Tablet and Capsules

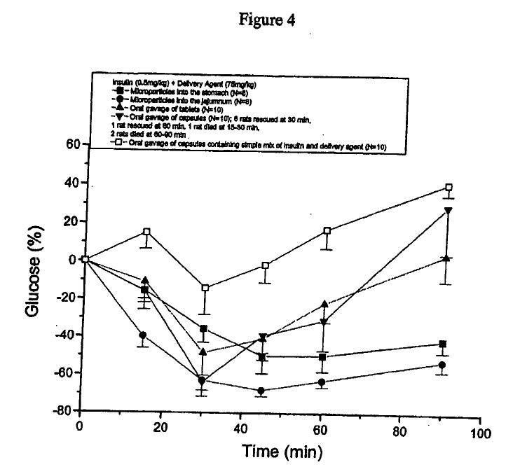

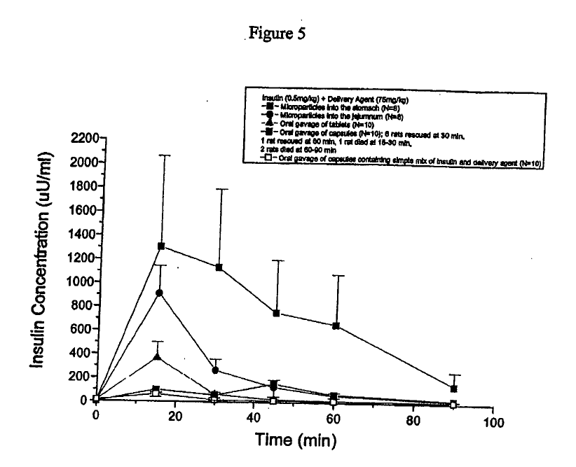

The glucose and insulin data from the three formulations tested are shown in FIGS. 4 and 5, respectively. The individual data are listed in Tables 6 to 7. The results from the direct dosing studies to the stomach and jejunum are included for comparison. The individual glucose and insulin data for the simple mix of insulin and delivery agent is shown in Table 8.

In the group of 10 rats that was dosed with capsules containing microparticles of coprocessed insulin and carrier, the average minimum glucose lowering was 70% from baseline at 30 minutes. One rat died at 15-30 minutes, likely due to hypoglycemia, six rats were rescued at 30 minutes with dextrose, an additional rat was rescued at 60 minutes, and two of the six that were rescued at 30 minutes died after 60 minutes. There were no signs of GI irritation or GI damage from the oral gavage procedure from necropsies of the rats after the experiment. The average minimum glucose lowering from tablets that contained the same amounts of insulin and carrier was 50%.

The corresponding insulin concentrations are shown in FIG. 5. Insulin concentration is highest from the coprocessed microparticles in a capsule, followed by the tablet and the capsule of the simple mix.

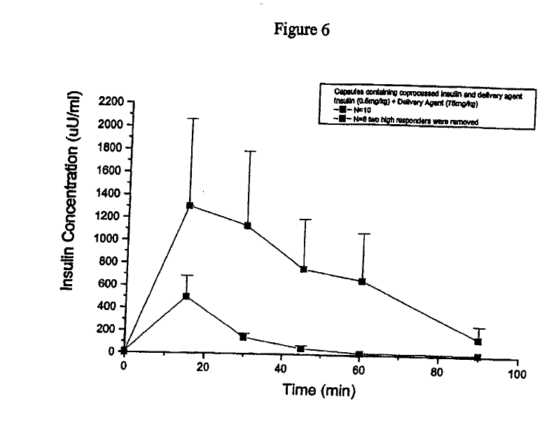

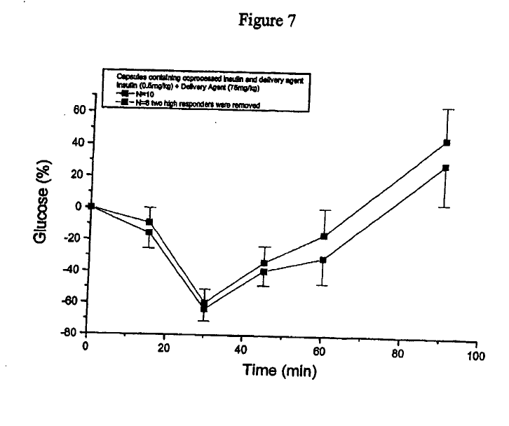

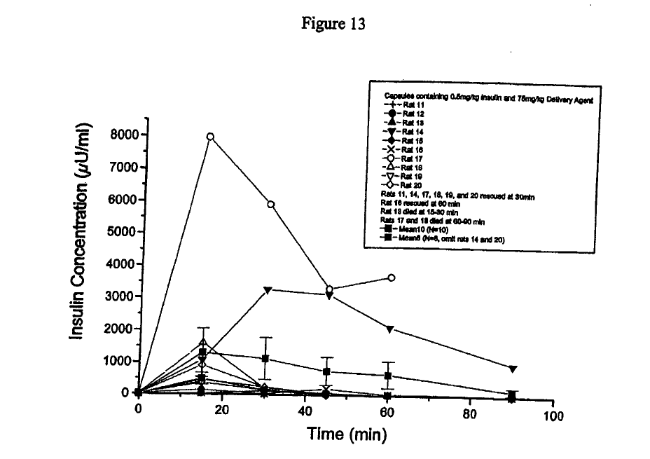

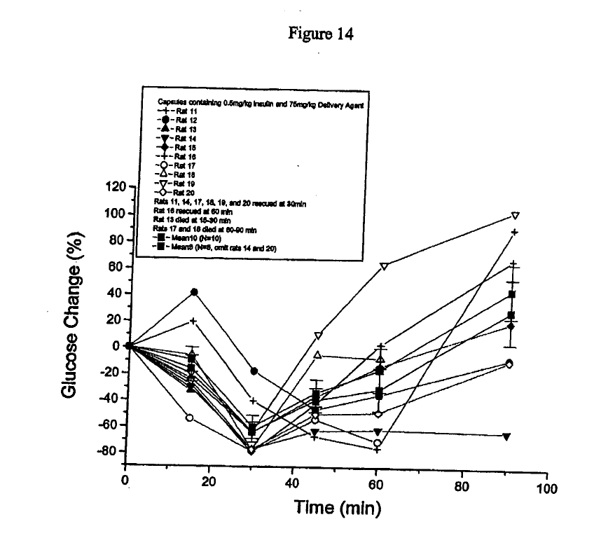

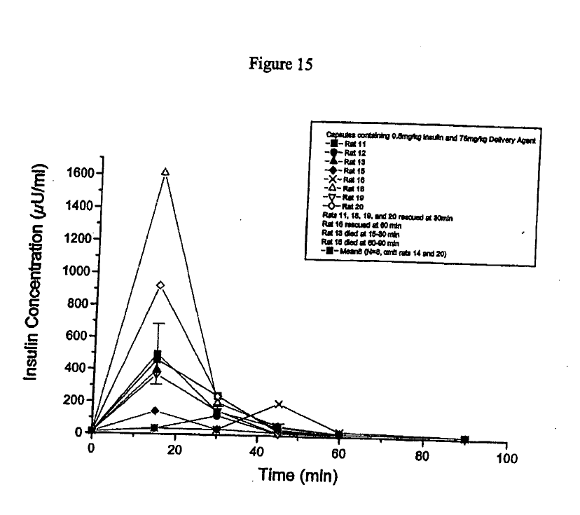

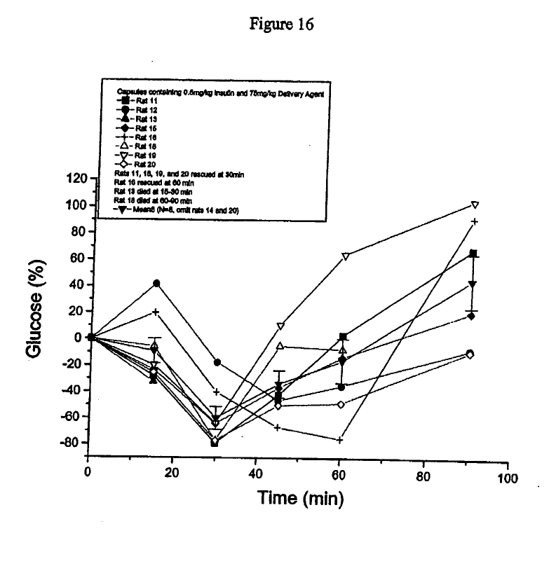

In the oral gavage studies using capsules containing coprocessed microparticles, two (of 10) rats were found to exhibit high insulin absorption. Retainer samples were reassayed and insulin levels were approximately the same as those from the original samples, as shown in Table 6(3), shown above. Insulin levels with and without two high responders are shown in FIGS. 6 and 7, respectively. The individual and average insulin and glucose profiles from N=10 and N=8 are shown in FIGS. 13 to 16.

TABLE 6
Oral gavage of tablets: Insulin (0.5 mg/kg), Delivery Agent (75 mg/kg)
1) Insulin

| | Rat | | | | | | | | | | | | | |
|---|---|---|---|---|---|---|---|---|---|---|---|---|---|---|
| Time (min) | #11 | #12 | #13 | #14 | #15 | #16 | #17 | #18 | #19 | #20 | mean | SD | SEM | CV |
| 0 | 12.5 | 12.5 | 12.5 | 12.5 | 12.5 | 12.5 | 12.5 | 12.5 | 12.8 | 12.5 | 12.5 | 0.1 | 0.0 | 0.8% |
| 15 | 468.8 | 162.1 | 700.1 | 12.5 | 1363.4 | 197.4 | 565.4 | 57.0 | 114.4 | 12.5 | 365.4 | 426.3 | 134.8 | 116.7% |
| 30 | 90.5 | 14.5 | 108.6 | 12.5 | 174.0 | 14.4 | 62.7 | 117.5 | 20.0 | 16.1 | 63.1 | 57.2 | 18.1 | 90.7% |
| 45 | 15.2 | 32.0 | 22.9 | 12.5 | 44.5 | 12.5 | 16.3 | 43.3 | 12.5 | 12.5 | 22.4 | 12.9 | 4.1 | 57.6% |
| 60 | 12.5 | 12.5 | 13.9 | 12.5 | 23.2 | 16.7 | 12.5 | 12.5 | 12.5 | 12.5 | 14.1 | 3.5 | 1.1 | 24.6% |
| 90 | 12.5 | 12.5 | 12.5 | 12.5 | 12.5 | 12.5 | 12.5 | 12.5 | 12.5 | 12.5 | 12.5 | 0.0 | 0.0 | 0.0% |
| AUC_{0→90} | 9180 | 3692 | 13068 | 1125 | 24532 | 4022 | 10229 | 3830 | 2768 | 1179 | 7363 | 7259 | 2295 | 98.6% |

2) Glucose

| Change | | | | | | | | | | | | | | |
|---|---|---|---|---|---|---|---|---|---|---|---|---|---|---|
| from | | | | | | | | | | | | | | |
| baseline | Rat | | | | | | | | | | | | | |
| Time (min) | # 1 | #2 | #3 | #4 | #5 | #6 | #7 | #8 | #9 | #10 | mean | SD | SEM | CV |
| 0 | 0 | 0 | 0 | 0 | 0 | 0 | 0 | 0 | 0 | 0 | 0 | 0 | 0.0 | 0.00% |
| 15 | −51.4 | −38.9 | −36.5 | −37.8 | −23.8 | −26.7 | 12.1 | 16.5 | 25.0 | 54.3 | −10.7 | 35.0 | 11.1 | −326.7% |
| 30 | −70.8 | −69.4 | −66.2 | −68.3 | −67.9 | −68.9 | −57.1 | −41.8 | −20.7 | 47.8 | −48.3 | 37.4 | 11.8 | −77.4% |
| 45 | −66.7 | −63.9 | −64.9 | −57.3 | −57.1 | −52.2 | −44.0 | −27.5 | −27.2 | 52.2 | −40.9 | 35.7 | 11.3 | −87.4% |
| 60 | −54.2 | −47.2 | −41.9 | −42.7 | −34.5 | −33.3 | −9.9 | −4.4 | −1.1 | 51.1 | −21.8 | 31.7 | 10.0 | −145.1% |
| 90 | −50.0 | −26.4 | −17.6 | −19.5 | −13.1 | 5.6 | 9.9 | 11.0 | 32.6 | 106.5 | 3.9 | 42.9 | 13.6 | 1099.9% |

TABLE 7
Oral gavage of capsules containing coprocessed insulin and delivery agent data
(Insulin (0.5 mg/kg), Delivery Agent (75 mg/kg))
1) Insulin

| Time | Rat | | | | | | | | | | | | | |
|---|---|---|---|---|---|---|---|---|---|---|---|---|---|---|
| (min) | #11 | #12 | #13 | #14 | #15 | #16 | #17 | #18 | #19 | #20 | mean | SD | SEM | CV |
| 0 | 12.5 | 12.5 | 12.5 | 12.5 | 12.5 | 12.5 | 12.5 | 12.5 | 12.5 | 12.5 | 0.0 | 0.0 | 0.0 | 0.0% |
| 15 | 461.1 | 35.2 | 400.0 | 1080.6 | 143.1 | 36.7 | 7970.5 | 1611.3 | 369.8 | 922.5 | 1303.1 | 2396.7 | 757.9 | 183.9% |
| 30 | 244.0 | 120.0 | | 3268.4 | 35.3 | 32.7 | 5915.1 | 201.0 | 150.9 | 240.3 | 1134.2 | 2070.4 | 654.7 | 182.5% |
| 45 | 26.9 | 13.7 | | 3132.7 | 18.9 | 201.2 | 3309.5 | 58.7 | 12.5 | 38.3 | 756.9 | 1399.0 | 442.4 | 184.8% |
| 60 | 13.8 | 12.5 | | 2129.7 | 12.5 | 28.7 | 3693.9 | 16.0 | 12.5 | 12.5 | 659.1 | 1335.7 | 422.4 | 202.6% |
| 90 | 12.5 | 12.5 | | 984.9 | 12.5 | 12.5 | | | 12.5 | 12.5 | 151.4 | 367.5 | 116.2 | 242.7% |
| AUC_{0→90} | 11752 | 3096 | | 175011 | 3522 | 4986 | 285725 | 28279 | 8561 | 18579 | 59946 | 100827 | 33609 | 168.2% |
| (n = 9) | | | | | | | | | | | | | | |
| AUC_{0→90} | 11752 | 3096 | | | 3522 | 4986 | | 28279 | 8561 | 18579 | 11254 | 9279 | 3507 | 82.4% |
| (n = 7) | | | | | | | | | | | | | | |

2) Glucose

| Time (min) | #11 | #12 | #13 | #14 | #15 | #16 | #17 | #18 | #19 | #20 | mean | SD | SEM | CV |
|---|---|---|---|---|---|---|---|---|---|---|---|---|---|---|
| 0 | 0 | 0 | 0 | 0 | 0 | 0 | 0 | 0 | 0 | 0 | 0 | 0 | 0.0 | 0.0% |
| 15 | −27.9 | 41.6 | −32.5 | −30.4 | −23.3 | 19.8 | −53.9 | −5.5 | −19.6 | −25.3 | −15.7 | 27.7 | 9.8 | −176.3% |
| 30 | −79.0 | −17.5 | | −76.6 | −64.0 | −40.1 | −78.0 | −77.0 | −63.8 | −77.0 | −63.7 | 21.4 | 7.5 | −33.5% |
| 45 | −42.1 | −46.4 | | −62.6 | −36.7 | −66.7 | −53.9 | −4.9 | 10.9 | −50.0 | −39.2 | 25.9 | 9.1 | −66.2% |
| 60 | 3.2 | −34.9 | | −61.4 | −13.3 | −75.3 | −70.3 | −7.2 | 64.5 | −48.3 | −30.8 | 45.9 | 16.2 | −149.0% |
| 90 | 68.4 | −7.2 | | −62.6 | 20.7 | 92.0 | | | 105.1 | −8.6 | 29.3 | 70.0 | 24.7 | 239.0% |

3) Reassay insulin levels of Rats 14 and 17

| | | |
|---|---|---|
| Time (min) | Insulin level (μU/ml), reassay | Insulin level (μU/ml), original |

Rat #14

| | | |
|---|---|---|
| 0 | 12.5 | 12.5 |
| 15 | 1020.8 | 1080.6 |
| 30 | 3018.0 | 3268.4 |
| 45 | 2590.5 | 3132.7 |
| 60 | 1714.3 | 2129.7 |
| 90 | 996.9 | 984.9 |

Rat #17

| | | |
|---|---|---|
| 0 | 12.5 | 12.5 |
| 15 | 7409.9 | 7970.5 |
| 30 | 6281.1 | 5915.1 |
| 45 | 2794.8 | 3309.5 |
| 60 | 2906.4 | 3693.9 |
| 90 | NS | NS |

TABLE 8
Oral gavage of capsules containing containing a simple mix of insulin and delivery agent data
(Insulin (0.5 mg/kg), Delivery Agent (75 mg/kg))
1) Insulin

rat

| time | #1 | #2 | #3 | #4 | #5 | #6 | #7 | #8 | #9 | #10 | mean | SD | SE | CV |
|---|---|---|---|---|---|---|---|---|---|---|---|---|---|---|
| 0 | 12.5 | 12.5 | 12.5 | 12.5 | 12.5 | 12.5 | 12.5 | 12.5 | 12.5 | 12.5 | 12.5 | 0 | 0 | 0.0% |
| 15 | 41.4 | 230.9 | 58.3 | 110.7 | 82.2 | 12.5 | 12.5 | 12.5 | 14.9 | 25.2 | 60.1 | 68.8 | 21.8 | 114.4% |
| 30 | 12.5 | 49.6 | 12.5 | 12.5 | 12.5 | 12.5 | 12.5 | 12.5 | 12.5 | 12.5 | 16.2 | 11.7 | 3.7 | 72.3% |
| 45 | 12.5 | 12.5 | 12.5 | 12.5 | 12.5 | 12.5 | 12.5 | 12.5 | 12.5 | 12.5 | 12.5 | 0 | 0 | 0.0% |
| 60 | 12.5 | 12.5 | 12.5 | 12.5 | 12.5 | 12.5 | 12.5 | 12.5 | 12.5 | 12.5 | 12.5 | 0 | 0 | 0.0% |
| 90 | 12.5 | 12.5 | 12.5 | 12.5 | 12.5 | 12.5 | 12.5 | 12.5 | 12.5 | 12.5 | 12.5 | 0 | 0 | 0.0% |
| AUC_{0→90} | 1559 | 4958 | 1812 | 2598 | 2171 | 1125 | 1125 | 1125 | 1161 | 1316 | 1895 | 1189 | 376 | 62.7% |

2) Glucose

| Change | | | | | | | | | | | | | | |
|---|---|---|---|---|---|---|---|---|---|---|---|---|---|---|
| from | | | | | | | | | | | | | | |
| base | | | | | | | | | | | | | | |
| line | Rat | | | | | | | | | | | | | |
| Time (min) | #1 | #2 | #3 | #4 | #5 | #6 | #7 | #8 | #9 | #10 | mean | SD | SEM | CV |
| 0 | 0 | 0 | 0 | 0 | 0 | 0 | 0 | 0 | 0 | 0 | 0 | 0 | 0 | 0% |
| 15 | 7.7 | −32.3 | 3.4 | 9.3 | 14.5 | 58.7 | 34 | 1.7 | 51.7 | 1.7 | 15.0 | 26.7 | 8.4 | 177.7% |
| 30 | −38.2 | −78.5 | −46.1 | −42.9 | −47.2 | 28.7 | 45.7 | 54.6 | 2.7 | −17.9 | −13.9 | 44.9 | 14.1 | −322.4% |
| 45 | −13.9 | −22.6 | −20.8 | −34.8 | −38.9 | 20.4 | 42.1 | 44.8 | −0.7 | 11.0 | −1.3 | 30.1 | 9.5 | −2247.0% |
| 60 | −3.1 | 41.4 | −18.5 | −14.3 | −26.9 | 20.9 | 65.5 | 47.7 | 26.2 | 32.9 | 17.2 | 31.3 | 9.9 | 182.1% |
| 90 | 6.2 | 73.1 | 32.6 | 53.4 | | 40.1 | 43.1 | 51.7 | 39.6 | 30.6 | 41.2 | 18.4 | 5.8 | 44.6% |

### Example 2

Summary of Intravenous, Portal Vein and Subcutaneous Experiments

a. Experiment

Intravenous, intraportal and subcutaneous dosing in rodents were conducted to estimate the absolute bioavailability, the absorption of insulin in the portal vein, and the bioavailability to relative subcutaneous administration. The data are summarized in Tables 9 to 11. The average insulin AUC_{0→∞}/Dose was 0.0093 min.kg/ml from intravenous dosing. This value was assumed to be constant in the estimates of absolute bioavailability.

TABLE 9
Insulin pharmacokinetics results from intravenous (IV) administration

| | | | AUC_{0->∞} | AUC_{0->∞}/ | | | | AUC_{0->∞}/ |
|---|---|---|---|---|---|---|---|---|
| Dose | Cₒ | kₑₗ | (min. | Dose | V_{d} | | C_{0->∞}/Dose | Dose |
| (μg/kg) | (ng/ml) | (min⁻¹) | ng/ml) | (min. kg/ml) | (ml/kg) | | (kg/ml) | (min. kg/ml) |
| 2.5 | 15.79 | 0.89 | 17.74 | 0.0071 | 158 | | 0.0063 | 0.0071 |
| 2.5 | 7.48 | 0.71 | 10.56 | 0.0042 | 334 | | 0.0030 | 0.0042 |
| 2.5 | 13.36 | 0.84 | 15.87 | 0.0063 | 187 | | 0.0053 | 0.0063 |
| 2.5 | 12.78 | 0.72 | 17.73 | 0.0071 | 196 | | 0.0051 | 0.0071 |
| 2.5 | 11.26 | 0.72 | 15.75 | 0.0063 | 222 | | 0.0045 | 0.0063 |
| X ± SD | 12.1 ± 2.8 | 0.78 ± 0.07 | 15.5 ± 2.6 | 0.0062 ± 0.0011 | 219 ± 68 | | | |
| (n = 5) | | | | | | | | |
| 8 | 50.67 | 0.51 | 99.35 | 0.0124 | 157.88 | | 0.0063 | 0.0124 |
| 8 | 52.64 | 0.53 | 99.32 | 0.0124 | 151.98 | | 0.0066 | 0.0124 |
| 8 | 49.63 | 0.48 | 103.4 | 0.0129 | 161.19 | | 0.0062 | 0.0129 |
| X ± SD | 51.0 ± 1.5 | 0.51 ± 0.03 | 100.7 ± 2.4 | 0.0126 ± 0.0003 | 157 ± 5 | | | |
| (n = 3) | | | | | | | | |
| 9 | 46.79 | 0.47 | 99.55 | 0.0111 | 192.35 | | 0.0052 | 0.0111 |
| 9 | 36.32 | 0.48 | 75.67 | 0.0084 | 247.80 | | 0.0040 | 0.0084 |
| 9 | 55.30 | 0.45 | 122.89 | 0.0137 | 162.75 | | 0.0061 | 0.0137 |
| X ± SD | 46.1 ± 9.5 | 0.47 ± 0.02 | 99.4 ± 23.6 | 0.0110 ± 0.0026 | 201 ± 43 | | | |
| (n = 3) | | | | | | | | |
| | | | | | | Average | 0.0053 | 0.0093 |
| | | | | | | SD | 0.001133858 | 0.0033 |
| | | | | | | SEM | 0.000341871 | 0.00099 |

TABLE 10
Insulin results from portal and systemic administrations

| n | AUC-IP | AUC-JV | AUC ratio | FPE (fH) |
|---|---|---|---|---|
| 1 | 9.95 | 17.74 | 0.56 | 0.44 |
| 2 | 6.21 | 10.56 | 0.59 | 0.41 |
| 3 | 8.88 | 15.87 | 0.56 | 0.44 |
| 4 | 14.51 | 17.73 | 0.82 | 0.18 |
| 5 | 8.69 | 15.75 | 0.55 | 0.45 |
| Average | 9.65 | 15.53 | 0.62 | 0.38 |
| SD | 3.04 | 2.94 | 0.12 | 0.12 |

TABLE 11
Insulin results from subcutaneous (SC) administration

| | | | | | | | | | | | | | | AUC_{0->T}/ |
|---|---|---|---|---|---|---|---|---|---|---|---|---|---|---|
| | Insulin | | | | | | | | | AUC_{0->T} | | | AUC_{0->T} | Dose |
| | dose | | Insulin | | | Glucose | | | | (min. | Tₘₐₓ | | (min. | (min. |
| Study # | (mg/kg) | Time | (uU/ml) | SD | SE | (mg/dl) | SD | SE | N | uU/ml) | (min) | Cₘₐₓ | ng/ml) | kg/ml) |
| 1 | 0.025 | 0 | 0.49 | 0.77 | 0.31 | 121 | 12.6 | 5.1 | 6 | 3640.8 | 15 | 76.1 | 140.0 | 0.005601 |
| | | 15 | 76.14 | 17.52 | 7.15 | 110 | 26.4 | 10.7 | 6 | | | | | |
| | | 30 | 54.34 | 21.92 | 8.95 | 90 | 31.4 | 12.8 | 6 | | | | | |
| | | 45 | 32.26 | 4.59 | 1.87 | 101 | 36.6 | 14.9 | 6 | | | | | |
| | | 60 | 21.61 | 4.29 | 1.75 | 102 | 30.4 | 12.4 | 6 | | | | | |
| | | 120 | 5.61 | 2.93 | 1.19 | 130 | 37.0 | 15.1 | 6 | | | | | |
| | | 180 | 1.64 | 1.82 | 0.74 | 194 | 54.7 | 22.3 | 6 | | | | | |
| 2 | 0.025 | 0 | 2.15 | 2.37 | 0.96 | 116 | 12.9 | 5.2 | 6 | 4234.4 | 15 | 79.5 | 162.9 | 0.006514 |
| | | 15 | 79.54 | 24.22 | 9.88 | 103 | 8.3 | 3.3 | 6 | | | | | |
| | | 30 | 53.03 | 20.27 | 8.27 | 90 | 17.4 | 7.1 | 6 | | | | | |
| | | 45 | 45.53 | 23.98 | 9.79 | 100 | 26.2 | 10.7 | 6 | | | | | |
| | | 60 | 23.48 | 21.47 | 8.76 | 120 | 23.6 | 9.6 | 6 | | | | | |
| | | 120 | 9.36 | 9.60 | 3.92 | 196 | 45.6 | 18.6 | 6 | | | | | |
| | | 180 | 3.48 | 5.23 | 2.13 | 188 | 45.1 | 18.4 | 6 | | | | | |
| 3 | 0.05 | 0 | 1.51 | 1.59 | 0.71 | 83 | 6.9 | 3.1 | 5 | 7230.0 | 15 | 98.8 | 278.1 | 0.005562 |
| | | 15 | 98.85 | 14.16 | 6.33 | 66 | 17.1 | 7.6 | 5 | | | | | |
| | | 30 | 73.59 | 25.49 | 11.40 | 60 | 21.4 | 9.5 | 5 | | | | | |
| | | 45 | 61.37 | 21.56 | 9.64 | 61 | 27.2 | 12.1 | 5 | | | | | |
| | | 60 | 58.20 | 32.09 | 14.35 | 73 | 24.4 | 10.9 | 5 | | | | | |
| | | 120 | 21.34 | 17.43 | 7.79 | 98 | 21.6 | 9.7 | 5 | | | | | |
| | | 180 | 8.30 | 7.49 | 3.35 | 105 | 30.7 | 13.7 | 5 | | | | | |
| 4 | 0.05 | 0 | 0.37 | 0.90 | 0.36 | 73 | 4.8 | 1.9 | 6 | 6407.7 | 15 | 103.4 | 246.5 | 0.004929 |
| | | 15 | 103.42 | 25.17 | 10.27 | 66 | 6.9 | 2.8 | 6 | | | | | |
| | | 30 | 81.64 | 24.94 | 10.18 | 59 | 10.5 | 4.3 | 6 | | | | | |
| | | 45 | 68.91 | 20.41 | 8.33 | 64 | 9.0 | 3.6 | 6 | | | | | |
| | | 60 | 58.58 | 21.87 | 8.93 | 72 | 7.0 | 2.8 | 6 | | | | | |
| | | 90 | 32.01 | 9.92 | 4.05 | 88 | 14.0 | 5.7 | 6 | | | | | |
| | | 120 | 21.15 | 9.42 | 3.84 | 110 | 17.0 | 6.9 | 6 | | | | | |
| 5 | 0.05 | 0 | 0 | 0 | 0 | 60 | 5.1 | 2.2 | 5 | 1317.0 | 15 | 34.9 | 50.7 | 0.001013 |
| | | 15 | 34.90 | 46.01 | 20.57 | 42 | 6.1 | 2.7 | 5 | | | | | |
| | | 30 | 9.71 | 18.15 | 8.12 | 38 | 6.9 | 3.1 | 5 | | | | | |
| | | 45 | 16.57 | 37.04 | 16.56 | 42 | 9.8 | 4.4 | 5 | | | | | |
| | | 60 | 10.46 | 20.05 | 8.96 | 48 | 17.6 | 7.8 | 5 | | | | | |
| | | 90 | 2.94 | 4.25 | 1.90 | 52 | 25.2 | 11.3 | 5 | | | | | |
| | | 120 | 5.05 | 6.91 | 3.09 | 66 | 41.8 | 18.7 | 5 | | | | | |
| 6 | 0.05 | 0 | 12.80 | 18.01 | 7.35 | 68 | 5.0 | 2.0 | 6 | 9937.5 | 15 | 191.7 | 382.2 | 0.007644 |
| | | 15 | 191.67 | 100.40 | 40.98 | 40 | 6.2 | 2.5 | 6 | | | | | |
| | | 30 | 119.71 | 79.53 | 35.56 | 39 | 2.5 | 1.1 | 5 | | | | | |
| | | 45 | 121.33 | 118.89 | 48.53 | 36 | 7.1 | 2.9 | 6 | | | | | |
| | | 60 | 74.65 | 57.97 | 23.66 | 32 | 4.3 | 1.7 | 6 | | | | | |
| | | 90 | 42.88 | 37.77 | 15.42 | 35 | 5.5 | 2.2 | 6 | | | | | |
| | | 120 | 25.65 | 17.59 | 7.18 | 39 | 9.2 | 3.7 | 6 | | | | | |
| 7 | 0.05 | 0 | 5.85 | 3.11 | 1.27 | 65 | 8.7 | 3.5 | 6 | 7711.1 | 30 | 126.0 | 296.6 | 0.005932 |
| | | 15 | 90.29 | 73.70 | 30.08 | 45 | 7.2 | 2.9 | 6 | | | | | |
| | | 30 | 125.96 | 107.54 | 43.90 | 39 | 8.9 | 3.6 | 6 | | | | | |
| | | 45 | 67.83 | 95.55 | 39.01 | 40 | 10.9 | 4.4 | 6 | | | | | |
| | | 60 | 78.19 | 105.69 | 43.14 | 42 | 13.2 | 5.4 | 6 | | | | | |
| | | 90 | 45.77 | 49.52 | 20.22 | 42 | 18.7 | 7.6 | 6 | | | | | |
| | | 120 | 18.24 | 13.53 | 5.52 | 50 | 26.2 | 10.7 | 6 | | | | | |
| 8 | 0.05 | 0 | 2.40 | 5.88 | 2.40 | 70 | 4.8 | 1.9 | 6 | 5303.3 | 15 | 131.7 | 204.0 | 0.004079 |
| | | 15 | 131.71 | 131.15 | 53.54 | 59 | 6.8 | 3.0 | 6 | | | | | |
| | | 30 | 70.90 | 45.95 | 18.76 | 64 | 12.1 | 4.9 | 6 | | | | | |
| | | 45 | 66.28 | 42.41 | 17.31 | 69 | 16.5 | 6.7 | 6 | | | | | |
| | | 60 | 0.59 | 1.27 | 0.52 | 73 | 13.1 | 5.3 | 6 | | | | | |
| | | 90 | 33.96 | 14.99 | 6.12 | 89 | 17.8 | 7.2 | 6 | | | | | |
| | | 120 | 14.64 | 9.35 | 3.82 | 106 | 18.6 | 7.6 | 6 | | | | | |
| | | | | | | | | | | | | | Avg | 0.005159 |
| | | | | | | | | | | | | | AUC_{0->T}/ | |
| | | | | | | | | | | | | | Dose | |
| | | | | | | | | | | | | | (min. | |
| | | | | | | | | | | | | | kg/ml) | |

Results

The ratio of systemic to portal insulin was found to be approximately 0.62 calculated from data in Table 10). Hence, the bioavailability in the portal vein can be calculated by dividing the absolute bioavailability by 0.62. The portal bioavailability provides an estimate of drug absorption from oral delivery. The average insulin AUC_{0→t}/Dose was 0.00516 min.kg/ml from subcutaneous dosing. This value is used to estimate bioavailability relative to subcutaneous. With the exception of the intravenous data, all AUC were calculated from t=0 to the last sampling point (i.e. AUC_{0→t}).

In the rat model, these results from intraportal administration suggest that the maximum absolute bioavailability of insulin is approximately 60% from oral delivery or by any other means of 100% GI absorption of insulin into the portal vein. Secondly, the absolute bioavailability from SC is approximately 56%.

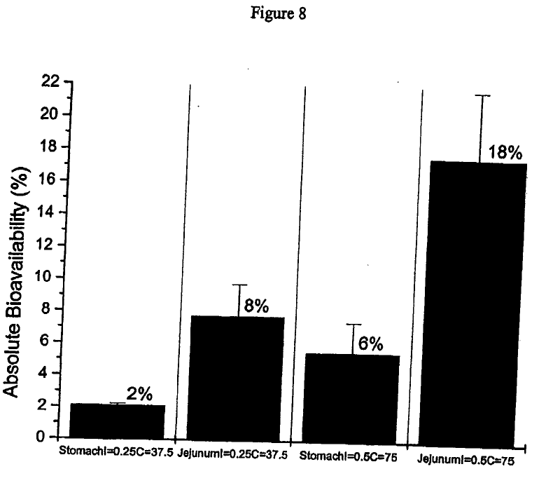

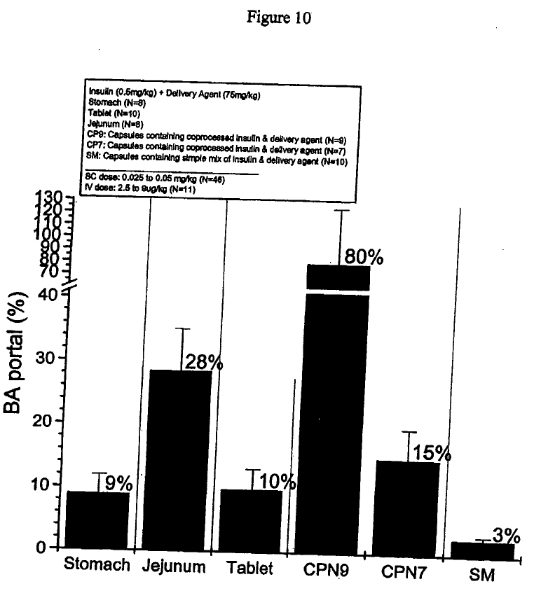

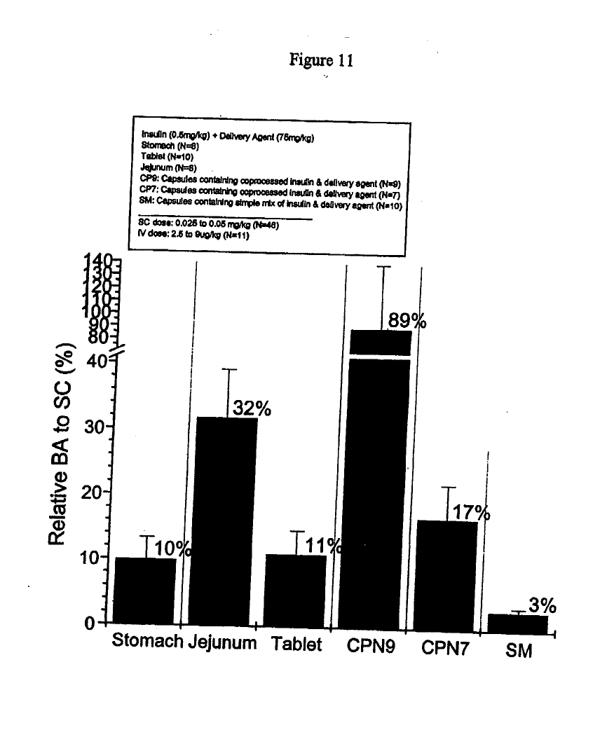

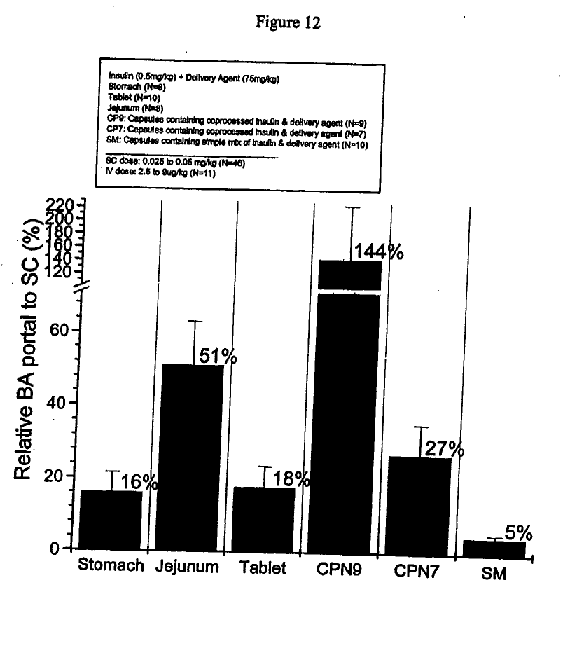

The estimates of bioavailability (absolute bioavailability, portal bioavailability, relative bioavailability to subcutaneous, and relative portal bioavailability to subcutaneous) are summarized in FIGS. 8 to 12, and in Table 12. The estimated absolute bioavailability from in situ dosing to the stomach and the jejunum are shown in FIG. 8. The values of bioavailability were 5% when dosed in the stomach and 18% when dosed in the jejunum from microparticles containing coprocessed insulin (0.5 mg/kg) and delivery agent (75 mg/kg).

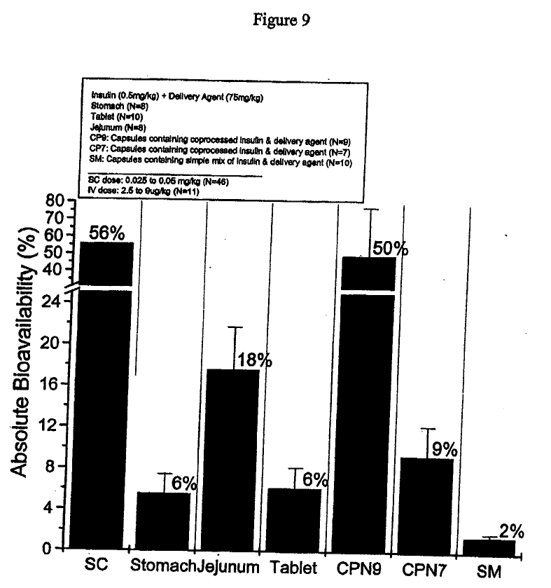

The estimated absolute bioavailability from the tablet and capsule formulations dosed by oral gavage in rats are shown in FIG. 9 using formulations containing 0.5 mg/kg insulin and 75 mg/kg delivery agent. The values of bioavailability were 6% when dosed from tablets, and 1.6% when dosed from capsules containing a simple mix of insulin and carrier.

TABLE 12
Estimates of bioavailability

| | | | | | AUC_{0->T}/ | | | Rel BA | Rel BAₚₒᵣₜₐₗ |
|---|---|---|---|---|---|---|---|---|---|
| | AUC_{0->T} | | | AUC_{0->T} | Dose | | BAₚₒᵣₜₐₗ | (%) | (%) |
| | (min. | Tₘₐₓ | Cₘₐₓ | (min. | (min. | BA | (%) | [AUC_{0->T}/ | [AUC_{0->T}/ |
| | uU/ml) | (min) | (uU/ml) | ng/ml) | kg/ml) | (%) | (IP/JV = 0.62) | Dose]sc = 0.0052 | Dose]sc = 0.0052 |
| SC | | | | | 0.0052 | 55.67 | | | |
| Insulin (0.5 mg/kg) + | | | | | | | | | |
| Delivery Agent (75 mg/kg) | | | | | | | | | |
| Stomach | 4778.70 | 15 | 114.50 | 183.80 | 3.68E−04 | 3.97 | 6.40 | 7.12 | 11.49 |
| | 2130.75 | 15 | 72.75 | 81.95 | 1.64E−04 | 1.77 | 2.85 | 3.18 | 5.12 |
| | 18958.58 | 45 | 893.86 | 729.18 | 1.46E−03 | 15.74 | 25.38 | 28.27 | 45.59 |
| | 1126.65 | 15 | 12.61 | 43.33 | 8.67E−05 | 0.94 | 1.51 | 1.68 | 2.71 |
| | 14425.95 | 45 | 212.73 | 554.84 | 1.11E−03 | 11.97 | 19.31 | 21.51 | 34.69 |
| | 4000.73 | 60 | 140.31 | 153.87 | 3.08E−04 | 3.32 | 5.36 | 5.96 | 9.62 |
| | 2793.45 | 15 | 118.66 | 107.44 | 2.15E−04 | 2.32 | 3.74 | 4.16 | 6.72 |
| | 5046.23 | 15 | 158.50 | 194.09 | 3.88E−04 | 4.19 | 6.76 | 7.52 | 12.13 |
| Mean | 6657.63 | 28.13 | 215.49 | 256.06 | 5.12E−04 | 5.53 | 8.91 | 9.93 | 16.01 |
| SD | 6446.07 | 18.70 | 280.36 | 247.93 | 4.96E−04 | 5.35 | 8.63 | 9.61 | 15.50 |
| SE | 2279.03 | 6.61 | 99.12 | 87.65 | 1.75E−04 | 1.89 | 3.05 | 3.40 | 5.48 |
| CV | 96.82 | 66.48 | 130.10 | 96.82 | 9.68E+01 | 96.82 | 96.82 | 96.82 | 96.82 |
| (%) | | | | | | | | | |
| Jejunum | 13504.88 | 15 | 413.23 | 519.42 | 1.04E−03 | 11.21 | 18.08 | 20.14 | 32.48 |
| | 21156.68 | 15 | 1193.25 | 813.72 | 1.63E−03 | 17.56 | 28.32 | 31.54 | 50.88 |
| | 11967.90 | 15 | 669.38 | 460.30 | 9.21E−04 | 9.93 | 16.02 | 17.84 | 28.78 |
| | 19689.45 | 15 | 1177.47 | 757.29 | 1.51E−03 | 16.34 | 26.36 | 29.36 | 47.35 |
| | 46001.85 | 15 | 2270.55 | 1769.30 | 3.54E−03 | 38.18 | 61.59 | 68.59 | 110.62 |
| | 11101.35 | 15 | 228.90 | 426.98 | 8.54E−04 | 9.21 | 14.86 | 16.55 | 26.70 |
| | 39190.05 | 15 | 954.35 | 1507.31 | 3.01E−03 | 32.53 | 52.47 | 58.43 | 94.24 |
| | 7234.05 | 15 | 374.88 | 278.23 | 5.56E−04 | 6.00 | 9.68 | 10.79 | 17.40 |
| Mean | 21230.78 | 15 | 910.25 | 816.57 | 1.63E−03 | 17.62 | 28.42 | 31.65 | 51.05 |
| SD | 14053.61 | 0 | 661.14 | 540.52 | 1.08E−03 | 11.66 | 18.81 | 20.95 | 33.80 |
| SE | 4968.70 | 0 | 233.75 | 191.10 | 3.82E−04 | 4.12 | 6.65 | 7.41 | 11.95 |
| CV | 66.19 | 0 | 72.63 | 66.19 | 6.62E+01 | 66.19 | 66.19 | 66.19 | 66.19 |
| (%) | | | | | | | | | |
| Tablet | 9181.20 | 15 | 468.81 | 353.12 | 7.06E−04 | 7.62 | 12.29 | 13.69 | 22.08 |
| | 3692.64 | 15 | 162.11 | 142.02 | 2.84E−04 | 3.07 | 4.94 | 5.51 | 8.88 |
| | 13068.89 | 15 | 700.10 | 502.65 | 1.01E−03 | 10.85 | 17.50 | 19.48 | 31.43 |
| | 1125.00 | 0 | 12.50 | 43.27 | 8.65E−05 | 0.93 | 1.51 | 1.68 | 2.71 |
| | 24533.57 | 15 | 1363.43 | 943.60 | 1.89E−03 | 20.36 | 32.84 | 36.58 | 59.00 |
| | 4022.33 | 15 | 197.39 | 154.70 | 3.09E−04 | 3.34 | 5.38 | 6.00 | 9.67 |
| | 10228.04 | 15 | 565.39 | 393.39 | 7.87E−04 | 8.49 | 13.69 | 15.25 | 24.60 |
| | 3830.06 | 30 | 117.47 | 147.31 | 2.95E−04 | 3.18 | 5.13 | 5.71 | 9.21 |
| | 2767.70 | 15 | 114.35 | 106.45 | 2.13E−04 | 2.30 | 3.71 | 4.13 | 6.66 |
| | 1178.43 | 30 | 16.06 | 45.32 | 9.06E−05 | 0.98 | 1.58 | 1.76 | 2.83 |
| Mean | 7362.78 | 16.50 | 371.76 | 283.18 | 5.66E−04 | 6.11 | 9.86 | 10.98 | 17.71 |
| SD | 7669.89 | 9.01 | 445.61 | 295.00 | 5.90E−04 | 6.37 | 10.27 | 11.44 | 18.44 |
| SE | 2425.43 | 2.85 | 140.91 | 93.29 | 1.87E−04 | 2.01 | 3.25 | 3.62 | 5.83 |
| CV | 104.17 | 54.63 | 119.86 | 104.17 | 1.04E+02 | 104.17 | 104.17 | 104.17 | 104.17 |
| (%) | | | | | | | | | |
| Capsule (co-dried) | 11570.64 | 15 | 461.06 | 445.02 | 8.90E−04 | 9.60 | 15.49 | 17.25 | 27.82 |
| (N = 9) | 3096.24 | 30 | 120.02 | 119.09 | 2.38E−04 | 2.57 | 4.15 | 4.62 | 7.45 |
| | 175011.18 | 30 | 3268.40 | 6731.20 | 1.35E−02 | 145.27 | 234.30 | 260.93 | 420.86 |
| | 3523.46 | 15 | 143.14 | 135.52 | 2.71E−04 | 2.92 | 4.72 | 5.25 | 8.47 |
| | 4988.01 | 45 | 201.24 | 191.85 | 3.84E−04 | 4.14 | 6.68 | 7.44 | 11.99 |
| | 285725.26 | 15 | 7970.50 | 10989.43 | 2.20E−02 | 237.16 | 382.52 | 426.00 | 687.10 |
| | 28278.49 | 15 | 1611.31 | 1087.63 | 2.18E−03 | 23.47 | 37.86 | 42.16 | 68.00 |
| | 8559.93 | 15 | 369.78 | 329.23 | 6.58E−04 | 7.11 | 11.46 | 12.76 | 20.58 |
| | 18578.54 | 15 | 922.50 | 714.56 | 1.43E−03 | 15.42 | 24.87 | 27.70 | 44.68 |
| Mean | 59925.75 | 21.67 | 1674.22 | 2304.84 | 0.00 | 49.74 | 80.23 | 89.35 | 144.11 |
| SD | 100838.27 | 10.90 | 2570.42 | 3878.39 | 0.01 | 83.70 | 135.00 | 150.34 | 242.49 |
| SE | 33612.76 | 3.63 | 856.81 | 1292.80 | 0.00 | 27.90 | 45.00 | 50.11 | 80.83 |
| CV | 168.27 | 50.29 | 153.53 | 168.27 | 168.27 | 168.27 | 168.27 | 168.27 | 168.27 |
| (%) | | | | | | | | | |
| Capsule (co-dried) | 11570.64 | 15 | 461.06 | 445.02 | 8.90E−04 | 9.60 | 15.49 | 17.25 | 27.82 |
| (N = 7) | 3096.24 | 30 | 120.02 | 119.09 | 2.38E−04 | 2.57 | 4.15 | 4.62 | 7.45 |
| | 3523.46 | 15 | 143.14 | 135.52 | 2.71E−04 | 2.92 | 4.72 | 5.25 | 8.47 |
| | 4988.01 | 45 | 201.24 | 191.85 | 3.84E−04 | 4.14 | 6.68 | 7.44 | 11.99 |
| | 28278.49 | 15 | 1611.31 | 1087.63 | 2.18E−03 | 23.47 | 37.86 | 42.16 | 68.00 |
| | 8559.93 | 15 | 369.78 | 329.23 | 6.58E−04 | 7.11 | 11.46 | 12.76 | 20.58 |
| | 18578.54 | 15 | 922.50 | 714.56 | 1.43E−03 | 15.42 | 24.87 | 27.70 | 44.68 |
| Mean | 11227.90 | 21.43 | 547.01 | 431.84 | 0.00 | 9.32 | 15.03 | 16.74 | 27.00 |
| SD | 9277.29 | 11.80 | 544.29 | 356.82 | 0.00 | 7.70 | 12.42 | 13.83 | 22.31 |
| SE | 3506.49 | 4.46 | 205.72 | 134.86 | 0.00 | 2.91 | 4.69 | 5.23 | 8.43 |
| CV | 82.63 | 55.08 | 99.50 | 82.63 | 82.63 | 82.63 | 82.63 | 82.63 | 82.63 |
| (%) | | | | | | | | | |
| Capsule (simple mix) | 1558.08 | 15 | 41.37 | 59.93 | 1.20E−04 | 1.29 | 2.09 | 2.32 | 3.75 |
| | 4956.66 | 15 | 230.89 | 190.64 | 3.81E−04 | 4.11 | 6.64 | 7.39 | 11.92 |
| | 1812.60 | 15 | 58.34 | 69.72 | 1.39E−04 | 1.50 | 2.43 | 2.70 | 4.36 |
| | 2598.35 | 15 | 110.72 | 99.94 | 2.00E−04 | 2.16 | 3.48 | 3.87 | 6.25 |
| | 2171.06 | 15 | 82.24 | 83.50 | 1.67E−04 | 1.80 | 2.91 | 3.24 | 5.22 |
| | 1125.00 | 0 | 12.50 | 43.27 | 8.65E−05 | 0.93 | 1.51 | 1.68 | 2.71 |
| | 1125.00 | 0 | 12.50 | 43.27 | 8.65E−05 | 0.93 | 1.51 | 1.68 | 2.71 |
| | 1125.00 | 0 | 12.50 | 43.27 | 8.65E−05 | 0.93 | 1.51 | 1.68 | 2.71 |
| | 1161.65 | 15 | 14.94 | 44.68 | 8.94E−05 | 0.96 | 1.56 | 1.73 | 2.79 |
| | 1315.97 | 15 | 25.23 | 50.61 | 1.01E−04 | 1.09 | 1.76 | 1.96 | 3.16 |
| Mean | 1894.94 | 10.50 | 60.12 | 72.88 | 1.46E−04 | 1.57 | 2.54 | 2.83 | 4.56 |
| SD | 1188.69 | 7.25 | 68.82 | 45.72 | 9.14E−05 | 0.99 | 1.59 | 1.77 | 2.86 |
| SE | 375.90 | 2.29 | 21.76 | 14.46 | 2.89E−05 | 0.31 | 0.50 | 0.56 | 0.90 |
| CV | 62.73 | 69.01 | 114.47 | 62.73 | 6.27E+01 | 62.73 | 62.73 | 62.73 | 62.73 |
| (%) | | | | | | | | | |
| Insulin (0.25 mg/kg) + | | | | | | | | | |
| Delivery Agent (37.5 mg/kg) | | | | | | | | | |
| Stomach | 1669.50 | 45 | 48.80 | 64.21 | 2.57E−04 | 2.77 | 4.47 | 4.98 | 8.03 |
| | 1249.50 | 15 | 20.80 | 48.06 | 1.92E−04 | 2.07 | 3.35 | 3.73 | 6.01 |
| | 1373.25 | 0 | 41.20 | 52.82 | 2.11E−04 | 2.28 | 3.68 | 4.09 | 6.60 |
| | 1192.50 | 0 | 21.50 | 45.87 | 1.83E−04 | 1.98 | 3.19 | 3.56 | 5.74 |
| | 1125.00 | 0 | 12.50 | 43.27 | 1.73E−04 | 1.87 | 3.01 | 3.35 | 5.41 |
| | 1125.00 | 0 | 12.50 | 43.27 | 1.73E−04 | 1.87 | 3.01 | 3.35 | 5.41 |
| | 1333.22 | 15 | 26.38 | 51.28 | 2.05E−04 | 2.21 | 3.57 | 3.98 | 6.41 |
| | 1125.00 | 0 | 12.50 | 43.27 | 1.73E−04 | 1.87 | 3.01 | 3.35 | 5.41 |
| Mean | 1274.12 | 9.38 | 24.52 | 49.00 | 1.96E−04 | 2.12 | 3.41 | 3.80 | 6.13 |
| SD | 186.56 | 15.91 | 13.77 | 7.18 | 2.87E−05 | 0.31 | 0.50 | 0.56 | 0.90 |
| SE | 65.96 | 5.63 | 4.87 | 2.54 | 1.01E−05 | 0.11 | 0.18 | 0.20 | 0.32 |
| CV | 14.64 | 169.71 | 56.16 | 14.64 | 1.46E+01 | 14.64 | 14.64 | 14.64 | 14.64 |
| (%) | | | | | | | | | |
| Jejunum | 8260.50 | 15 | 428.50 | 317.71 | 1.27E−03 | 13.71 | 22.12 | 24.63 | 39.73 |
| | 10947.00 | 15 | 532.40 | 421.04 | 1.68E−03 | 18.17 | 29.31 | 32.64 | 52.65 |
| | 5229.00 | 15 | 232.60 | 201.12 | 8.04E−04 | 8.68 | 14.00 | 15.59 | 25.15 |
| | 1125.00 | 0 | 12.50 | 43.27 | 1.73E−04 | 1.87 | 3.01 | 3.35 | 5.41 |
| | 1910.94 | 15 | 61.95 | 73.50 | 2.94E−04 | 3.17 | 5.12 | 5.70 | 9.19 |
| | 3735.93 | 15 | 186.56 | 143.69 | 5.75E−04 | 6.20 | 10.00 | 11.14 | 17.97 |
| | 2157.20 | 15 | 79.60 | 82.97 | 3.32E−04 | 3.58 | 5.78 | 6.43 | 10.38 |
| | 3897.03 | 15 | 160.54 | 149.89 | 6.00E−04 | 6.47 | 10.43 | 11.62 | 18.74 |
| Mean | 4657.82 | 13.13 | 211.83 | 179.15 | 7.17E−04 | 7.73 | 12.47 | 13.89 | 22.40 |
| SD | 3392.58 | 5.30 | 182.48 | 130.48 | 5.22E−04 | 5.63 | 9.08 | 10.12 | 16.32 |
| SE | 1199.46 | 1.88 | 64.52 | 46.13 | 1.85E−04 | 1.99 | 3.21 | 3.58 | 5.77 |
| CV | 72.84 | 40.41 | 86.14 | 72.84 | 7.28E+01 | 72.84 | 72.84 | 72.84 | 72.84 |
| (%) | | | | | | | | | |

### Example 3

### Insulin and 4-CNAB Stability in Simulated Gastric Fluid

The stability of insulin in simulated gastric fluid (SGF) was evaluated in the presence and absence of 4-CNAB. Solutions were prepared containing insulin (1 mg/ml) with and without monosodium 4-CNAB (1 mg/ml).

The SGF was prepared with and without pepsin, a gastric enzyme. SGF pH 1.2 was prepared as per the USP NF 26 guidelines. 2 g sodium chloride and 3.2 g of pepsin were weighed and added to a suitable container, and deionized water was added to reach one liter in volume. If necessary, the pH was adjusted to 1.2 by addition of concentrated HCl or NaOH. A second SGF solution omitting the pepsin was also prepared.

Four 50 ml samples of SGF (two with pepsin and two without) were placed into a jacketed vessel connected to a circulating water bath set at 37° C. The solutions were stirred with magnetic stir bars for ten minutes to allow the solutions to reach 37° C. and reach thermal equilibrium. 50 mg of 4-CNAB was added to one of the samples containing pepsin and one of the samples without pepsin, and the solutions were stirred for a few minutes to allow the 4-CNAB to dissolve. 50 mg of insulin was added to the each of the samples. After dissolution of the insulin, samples of the solutions were taken at pre-determined time intervals, filtered, and immediately assayed by HPLC for insulin and 4-CNAB content. The first sample withdrawn after all the insulin was dissolved was considered to have been drawn at time zero (0). The results are shown in table 13.

TABLE 13

| | | | | Insulin | Insulin | | |
|---|---|---|---|---|---|---|---|
| | | | | without 4- | without 4- | | |
| | | | | CNAB | CNAB | | |
| | | | | and with | and | | |
| | | Insulin with 4-CNAB | | Enzymes | without | Insulin with 4-CNAB | |
| | | and Enzymes (pepsin) | | (pepsin) | Enzymes | and no Enzymes | |
| | | | 4-CNAB | Insulin % | Insulin % | Insulin % | 4-CNAB |
| | | Insulin % of | % of | of | of | of | % of |
| Time | | theoretical | theoretical | theoretical | theoretical | theoretical | theoretical |
| 0 | minutes | 3.0 | 105.6 | 3.0 | 99.8 | 100.8 | 95.3 |
| 10 | minutes | | | | 100.4 | 103.9 | 95.7 |
| 20 | minutes | | | | 100.6 | 103.8 | 99.6 |
| 30 | minutes | | | | 100.1 | 105.7 | 99.2 |
| 1 | h | | | | 99.4 | 97.8 | 94.8 |
| 2 | h | | | | 99.0 | 102.2 | 95.8 |
| 24 | h | | | | 0 | 0 | 91.2 |

The term “% of theoretical” as used herein, means the percent of the concentration (mg/mL) of withdrawn solution at the time-point the sample was taken as compared to the theoretical concentration (mg/mL) of the measuring component for experiment. The standard of deviation for the HPLC analysis is ±5%. These results show that insulin is unstable in SGF containing pepsin, since only 3.0% of the insulin remained at the first sampling point (97% of the insulin was degraded), while insulin is stable at least up to 2 hours in SGF without pepsin.

### Example 4

### Stability of Insulin in Simulated Intestinal Fluid

The stability of insulin in simulated intestinal fluid (SIF) was evaluated in the presence and absence of 4-CNAB.

The SIF solutions were prepared with and without pancreatic enzyme. SIF pH 7.5 was prepared as per the USP NF 26 guidelines. SIF was prepared by addition of 6.8 g monobasic potassium phosphate and 10 g of pancreatin into a suitable vessel, and deionized water was added to reach a total volume of one liter. If necessary, the pH was adjusted to 7.5 by addition of 0.2 N sodium hydroxide. A second SIF solution omitting the pancreatin, an intestinal enzyme, was also prepared.

Four 50 ml samples of SIF (two with pancreatin and two without) were placed into a jacketed vessel connected to a circulating water bath set at 37° C. The solutions were stirred with magnetic stir bars for ten minutes to allow the solutions to reach 37° C. and reach thermal equilibrium. 50 mg of 4-CNAB was added to one of the samples containing pepsin and one of the samples without pepsin, and the solutions were stirred for a few minutes to allow the 4-CNAB to dissolve. 50 mg of insulin was added to the each of the samples. After dissolution of the insulin, samples of the solutions were taken at pre-determined time intervals, and immediately assayed by HPLC for insulin and 4-CNAB content. The results are shown in table 14.

TABLE 14

| | | | | | Insulin | | |
|---|---|---|---|---|---|---|---|
| | | | | | without 4- | | |
| | | | | Insulin | CNAB and | | |
| | | Insulin with 4-CNAB | | without 4- | with | | |
| | | and Enzymes | | CNAB or | Enzymes | Insulin with 4-CNAB | |
| | | (pancreatin) | | Enzymes | (pancreatin) | and no Enzymes | |
| | | Insulin % | 4-CNAB | Insulin % | 4-CNAB % | Insulin % | 4-CNAB |
| | | of | % of | of | of | of | % of |
| Time | | theoretical | theoretical | theoretical | theoretical | theoretical | theoretical |
| 0 | minutes | 58.9 | 98.6 | 92.9 | 66.9 | 100.2 | 103.9 |
| 2 | minutes | 26.9 | 98.9 | — | 55.2 | 102.1 | 105.4 |
| 4 | minutes | 19.3 | 99.0 | — | 45.1 | 106.3 | 111.5 |
| 6 | minutes | 5.4 | 98.8 | — | 34.5 | 103.1 | 106.0 |
| 8 | minutes | 3.1 | 98.6 | — | 24.5 | 101.7 | 101.8 |
| 10 | minutes | 2.5 | 98.2 | 92.5 | 15.1 | 101.3 | 103.4 |
| 15 | minutes | | | 92.7 | | 100.9 | 104.0 |
| 30 | minutes | | | 94.7 | | 100.6 | 104.0 |
| 45 | minutes | | | — | | 99.1 | 102.3 |
| 1 | hour | | | 92.7 | | 95.9 | 100.0 |
| 2 | hours | | | 94.6 | | — | — |
| 24 | hours | | | 100.4 | | 105.6 | 111.3 |

These results show that insulin is stable in SIF without pancreatin and degrades in presence of the enzyme. Insulin is more stable in SIF with and without enzyme than in SGF with and without enzyme. At the first sampling time point (0 minuts) only 3.0% insulin remained in SGF with enzymes while 58.9% and 66.9% insulin remained in SIF.

### Example 5

### Effect of Formulation on Insulin Absorption and Action

Six formulations containing insulin shown in Table 15 were prepared as follows.

TABLE 15

| ID | Formulation |
|---|---|
| 1 | Fast Disintegrating Tablets - 1. Insulin, 4-CNAB, 0.4% w/w |
| | povidone, 10% w/w Polyplasdone XL, 50.7% w/w Emcocel HD90, |
| | 1% w/w SLS and 1% w/w Magnesium Stearate |
| 2 | Fast Disintegrating Tablets - 2. Insulin, 4-CNAB, 0.4% w/w |
| | povidone 10% w/w Polyplasdone XL, 50.2% w/w Prosolv HD90, |
| | 1% w/w SLS and 1% w/w Magnesium Stearate |
| 3 | Tablets with Emcompress. Insulin, 4-CNAB, 0.4% w/w povidone, |
| | ˜29.1% w/w Emcompress, 1% w/w SLS and 1% w/w |
| | Magnesium Stearate |
| 4 | Tablets with Mg Stearate only. Insulin, 4-CNAB, 0.4% w/w |
| | povidone, and 1% w/w Magnesium Stearate |
| 5 | Tablets with Anhydrous Emcompress. Insulin, 4-CNAB, |
| | 0.4% w/w povidone, ˜29.1% w/w Anhydrous Emcompress, |
| | 1% w/w SLS and 1% w/w Magnesium Stearate |
| 6 | Co-dried capsules. Insulin and 4-CNAB were dissolved in water. |
| | The solution was co-dried and the resulting powder was filled into |
| | hard gelatin capsules. |

Polyplasdone XL, is available from International Specialty Products, Wilmington Del.; Emcocel HD90, Prosolv HD90, Emcompress and Anhydrous Emcompress is available from JRS Pharma, Patterson, N.Y.

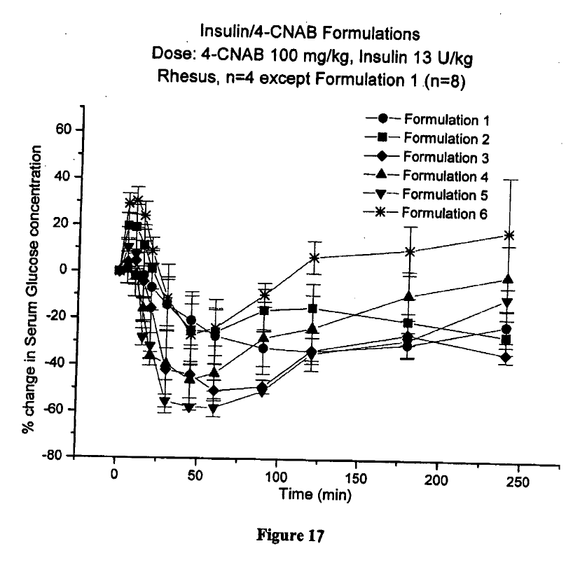

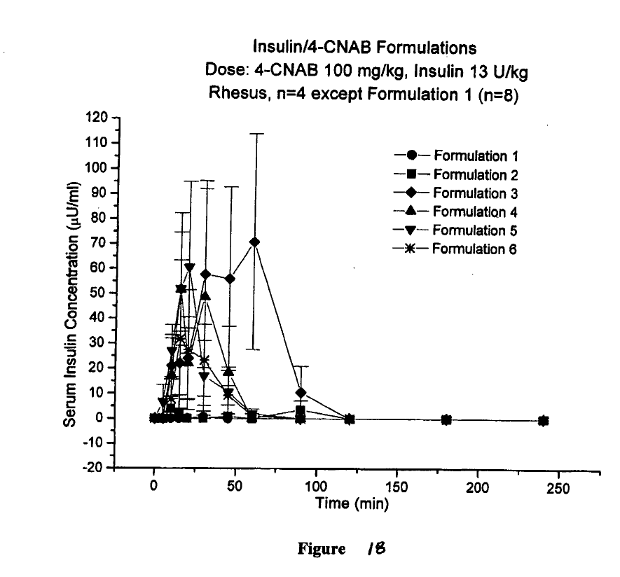

The formulations were fed to rhesus monkeys in doses containing 100 mg/kg of 4-CNAB and 13 U/kg insulin. Groups of four rhesus monkeys, two males and two females, were fasted for at least 12 hrs prior to dosing and up to 4 hrs after dosing. Water was withheld approximately 1 hr before dosing and up to 2 hrs after dosing after which it was permitted ad libitum. The dosing was followed by a 5 ml water flush. Blood samples (approximately 2 ml each) were collected by venipuncture at 15 minutes before dosing and at 5, 10, 15, 20, 30, 45 minutes and 1, 1.5, 2, 3, 4 hr after dosing. Each blood sample was divided into two portions. One portion was allowed to clot at room temperature and centrifuged at 2-8° C. for 10 minutes at 3000 rpm. The serum obtained was aliquoted into two portions and stored at −70° C. until shipment. One sample was shipped to Emisphere on dry ice for insulin analysis by ELISA while the other was retained by the CRO for serum glucose analysis. The second portion of the blood was kept on wet ice for up to 30 minutes and centrifuged at 2-8° C. for 10 minutes at 3000 rpm. The plasma obtained was shipped to Emisphere on dry ice for analysis of 4-CNAB content by HPLC. Each formulation was administered to 4 rhesus monkeys, except formulation 1, which was administered to 8 rhesus monkeys. Blood samples were taken at predetermined intervals as described above and assayed for insulin and glucose levels. The results are shown in table 16 and in FIGS. 17 and 18.

TABLE 16

| | | | | | Serum Glucose | |
|---|---|---|---|---|---|---|
| | | | | | tₘᵢₙ | |
| | Disint. | | Onset of glucose | Insulin | (Cmin, % change | Duration of Action |
| Form. ID | Time | | lowering | tₘₐₓ | from baseline ± SE) | (Glucose Level) |
| 1 | 35 | sec | 4 min | 30 min | 120 min | 240 min+ |
| | | | | | (−33.57 ± 8.25%) | (−21.87 ± 6.64%) |
| 2 | 23 | sec | 8 min | 10 min | 60 min | 240 min+ |
| | | | | | (−25.6 ± 13.91%) | (−26.17 ± 7.39%) |
| 3 | 6 | min | 7 min | 60 min | 60 min | 240 min+ |
| | 43 | sec | | | (−50.68 ± 4.41%) | (−33.96 ± 3.36%) |
| 4 | 8 | min | 2 min | 15 min | 45 min | 240 min |
| | 40 | sec | | | (−46.27 ± 7.78%) | (−0.80 ± 13.98%) |
| 5 | 7 | min | 6 min | 20 min | 60 min | 240 min+ |
| | 6 | sec | | | (−58.13 ± 3.89%) | (−10.14 ± 0%) |
| 6 | | | 9 min | 15 min | 45 min | 90 min |
| | | | | | (−26.96 ± 13.13%) | (18.43 ± 23.84%) |

Disintegration time was determined in water at 37±2° C. using the method described in USP <701>. Multiple tubes containing water are placed in a basket-rack assembly immersed in a water bath maintained at 37±2° C. The basket-rack assembly raises and lowers the tubes at a constant frequency. The tablets are placed in the tubes and are periodically examined to determine if they have disintegrated completely. Each tablet is tested in six different tubes. If 1 or 2 tablets fails to consistently disintegrate, the procedure is repeated on additional tablets. The average maximum concentration of insulin (Cₘₐₓ) was determined for each group based upon the serum levels of insulin measured as described above. If the blood glucose levels in the primates falls to very low levels (<1 mmol/L) during the experiment they are administered dextrose in order to bring the blood glucose up to a safe level. The average Cₘₐₓ for each group, as well as the number of rhesus monkeys rescued, is shown in table 17.

TABLE 17

| Formulation | Insulin Cₘₐₓ | No. of primates | No. of primates |
|---|---|---|---|
| ID | (μU/ml) | dosed | rescued |
| 1 | 0.6585 ± 0.6585 | 8 | 0 |
| 2 | 3.99 ± 3.99 | 4 | 0 |
| 3 | 70.81 ± 43.22 | 4 | 2 |
| 4 | 51.65 ± 30.69 | 4 | 1 |
| 5 | 60.46 ± 34.56 | 4 | 3 |
| 6 | 31.75 ± 31.54 | 4 | 1 |

### Example 6

### Preparation of Enteric Coated Tablets

Capsules were manufactured by encapsulating 300 mg of a formulation including 150 units insulin, 200 mg 4-CNAB, 0.4% w/w povidone, ˜29.1% w/w Emcompress, 1% w/w SLS, and 1% w/w/ magnesium stearate into size 2 white opaque capsules. The capsules were first coated with a subcoat consisting of Opadry clear for a weight gain of 5% followed by an enteric coat of 20% weight gain for a total weight gain on the capsules of 25%.

Tablets were manufactured by pressing 300 mg of the formulation described above into tablets. An 10% weight gain enteric coat was applied. The formulations for the subcoats and enteric coats are shown in table 18 below.

TABLE 18

| | Tablets | Capsules |
|---|---|---|
| Ingredients | % w/w | % w/w |
| SUBCOAT | | |
| Opadry Clear | NA | 8.0 |
| Milli Q Water | NA | 92.0 |
| Total | NA | 100.0 |
| ENTERIC COAT | | |
| Eudragit L30D55 | 49.4 | 49.4 |
| Talc | 3.7 | 3.7 |
| Triethyl Citrate | 1.5 | 1.5 |
| Milli Q Water | 45.4 | 45.4 |
| Total | 100.0 | 100.0 |

Opadry™ Clear is available from Colorcon, of West Point, Pa.

Milli Q Water is highly purified water and is available from Millipore of Billerica, Mass.

Eudragit L30D55 is available from Degussa AG, Parsippany, N.J.

To verify the effectiveness of the enteric coat, the coated capsules and tablets were placed in 0.1 N HCl for two hours or pH 6.8 phosphate buffer for one hour. The coated capsules and tablets did not dissolve in the 0.1 N HCl, but did dissolve in the pH 6.8 phosphate buffer.

### Example 7

### SNAC Micro Beads Coated With Heparin

5 g of SNAC and 0.5 g of magnesium stearate were mixed. 0.02 g of the mixed powder was fed into a die. Small beads of SNAC and magnesium stearate were made at 1200 PSI bar pressure The beads had a round/ball shape size of about 0.2 mm to about 2.0 mm. The SNAC beads were then coated with 2.5 g of heparin, in liquid form, by a rotary method and dried under vacuum oven at 40° C. for 10 hours.

### Example 8

### Micronized SNAD with Heparin

SNAD was screened through a 35 mesh Tyler standard sieve. The SNAD was milled with a Glen Mills, Model S100 centrifugal ball mill (Clifton, N.J.) equipped with a 250 mL stainless steel grinding jar and 30 mm (440 c) diameter stainless steel balls was used. The process parameters investigated were (1) number of balls used, (2) duration of milling, (3) milling speed, and (4) milling jar total charge. A Malvern Mastersizer 2000 equipped with a Scirocco 2000 dry accessory was used for particle size determination. A Kratos XRD 6000 (version 4.1) X-ray powder diffractometer scanning over the 2θ range 5-40° 2θ was used for monitoring crystallinity changes. The diverging, scattering, and receiving slits were 1°, 1°, and 0.3 mm respectively. A Brinkmann 737 KF coulometer was used for moisture content determination while a Quantachrome Nova 3000 Series Surface Area Analyzer was used for specific surface area determination.

The results indicated that the particle size distribution of pre-screened SNAD was d(0.1)=1.6 μm, d(0.5)=10.5 μm, and d(0.9)=314.9 μm. The data obtained using different numbers of balls ranging from 1 to 5 indicated that the optimum number of balls for the charge used was 2. The use of 2 balls yielded the particle size d(0.1)=1.1 μm, d(0.5)=12.0 μm, a d(0.9)=154.3 μm.

An evaluation of the effect of milling time for a fixed number of balls and charge indicated that a milling time of 120 minutes was optimum resulting in the particle size distribution, d(0.1)=2.0 μm, d(0.5)=15.4, and d(0.9)=62.9 μm.

An evaluation of the milling speeds 100, 300, and 500 rpm indicated that optimum milling was obtained at 300 rpm. This speed yielded the particle size distribution, d(0.9)=62.9 μm compared to unmilled SNAD d(0.9)=314.9 μm.

A charge of 37 mL of the 250 mL milling jar provided better milling compared to 75 and 112 mL. The powder X-ray diffraction analysis indicated that milling did not result in crystallinity changes for SNAD. The Karl Fischer moisture content determination indicated no significant changes in moisture content.

The SNAD was then mixed with heparin.

### Example 9

### Micronized SNAC with Micronized Heparin

SNAC and heparin were micronized separately by the procedure described in Example 8 with 2 balls at 200 rpm for 120 minutes and then mixed together. The micronized SNAC had a d(0.5) of 7.574 μm SNAC/heparin capsules having the formulations shown in table 19 below were prepared by hand packing them into hard gelatin capsules.

TABLE 19

Formulation
(mg/capsule)

| Ingredient | A | B |
|---|---|---|
| Micronized SNAC | 125 | 125 |
| Micronized Heparin USP | 158 | 158 |
| (30,000 U) | | |
| Propylene Glycol | 105 | 105 |
| Monocaprylate¹ | | |
| Sodium lauryl sulfate | 7 | 7 |
| PEG 300² | 305 | 270 |
| Water | — | 35 |
| Total | 700 | 700 |

¹Propylene glycol monocaprylate is available as Capmul ™ PG 8 from Abitec Corporation of Columbus, OH.
²PEG 300 is available as Carbowax ™ 300 from Dow Chemical Co. of Midland, MI.

The heparin, SNAC, and sodium lauryl sulfate were mixed. Separately, the PEG 300, propylene glycol monocaprylate, and water (for formulation B) were mixed. 50% of the liquid PEG 300/propylene glycol monocaprylate mixture was transferred to a mortar. The heparin, SNAC, and sodium lauryl sulfate blended powder was added little by little and triturated with the liquid in the mortar and pestle. The capsules were then packed with the resulting mixture.

### Example 10

### Micronized SNAC/Heparin

Heparin (118.5 mg/dose (22,500 rpm)) and SNAC (125 mg/dose) were dry mixed, screened through a 35 mesh screen, and milled for about 4 minutes with a ball mill. The mixture was packed into capsules (Capsugel Size 1 capsules (Greenwood, S.C.)).

The capsules were administered to rhesus monkeys (2 capsules per monkey) by the following procedure. Rhesus monkeys weighing between 3.5-5.0 kg were fasted overnight before the experiments and food was returned about 2 hours after dosing. Water was withheld from 30 minutes prior to dosing until 30 minutes after dosing, except for those quantities used for dosing. Each dosage form was delivered to the rear of the mouth using a pill gun. After release of the dosage form, 5 ml of reverse osmosis water was administered into the oral cavity to facilitate swallowing. Following delivery, the oral cavity was inspected to ensure that the capsule was swallowed. Antifactor Xa from blood samples was measured over 6 hours.

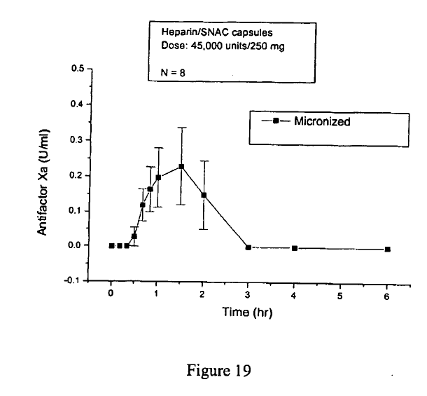

The results are shown in FIG. 19.

### Example 11

### Micronized SNAC/Heparin

Capsules containing micronized SNAC/heparin as shown in table 20 below were prepared as follows.

A solution of heparin and SNAC was prepared as follows. The required amounts of heparin and SNAC were weighed out and water, which was previously adjusted to a pH of about 8 with sodium hydroxide, was added. The pH of the resulting solution was in the range of about 7.3-7.5. The solution pH was adjusted to a pH of about 8 with sodium hydroxide. The solution was then dried in a RotoVap apparatus at 50° C. under vacuum. The evaporating was done using the program outlined below.

1. Immediate reduction of vacuum from 760 torr to 200 torr

2. Reduction of vacuum pressure from 200 to 100 torr in 2 minutes

3. Reduction of vacuum pressure from 100 to 50 torr in 2 minutes

4. Reduction of vacuum pressure from 50 to 25 torr in 4 minutes

5. Reduction of vacuum pressure from 25 to 15 torr in 4 minutes

6. Reduction of vacuum pressure from 15 to 10 torr in 2 minutes

7. Evaporating at 10±2 torr and 70 rpm in 30 minutes

8. Switch to 50 rpm manually and continue with evaporating for 4 hours

The sample was vacuum dried overnight. The resulting powder was then micronized and filled into capsules to give the desired dose.

TABLE 20

| | Formulation (mg/capsule) | | |
|---|---|---|---|
| Ingredient | A | B | C |
| Micronized SNAC | 198 | 173 | 210.6 |
| Micronized Heparin | 72 | 63 | 78 |
| USP | (13104 USP | (11466 USP | (14196 USP |
| | heparin units) | heparin units) | heparin units) |
| Total | 270 | 236 | 288.6 |

### Prophetic Example 12

Micronized 5-CNAC disodium and tablets of salmon calcitonin plus micronized 5-CNAC disodium may be prepared in accordance with the present invention as follows:

### Preparation of Micronized 5-CNAC Disodium

Coarse 5-CNAC disodium, which is to be micronized, is added to a jet mill (Air Jet Mill GemT®, Copley Scientific, Ltd., Nottingham, UK) using a 80 ceramic pan cake jet mill, 8 cm diameter, 6 bar N2, 0.5 mm nozzles with manual feed of about 700 g/h. The coarse 5-CNAC disodium is jet milled and periodically sampled under microscope with reference ruler measurements to identify when the average desired micronized particle size is obtained. Three different batches are ground to create 6 μm, 35 μm, and 46 μm batches. Individual sieving of the separate micronized batches is then done by using a conical sieve mill (Quadro Comil, Quadro Engineering Incorporated 613 Colby Drive, Waterloo, Ontario, Canada N2V 1A1) with a U10, 813 μm conical sieve, round beater, operating at 1500 upm with throughput of about 150 kg/h.

Formulation I
Salmon Calcitonin Formulation with 5-CNAC Disodium
of Different Particle Size

| Ingredient | Amount (mg) | Percent (%) |
|---|---|---|
| Salmon Calcitonin | 1 | 0.25 |
| Micronized 5-CNAC | 228 | 57 |
| Disodium | | |
| Avicel PH 102 ® | 147 | 36.75 |
| Crospovidone, NF | 20 | 5 |
| Magnesium stearate | 4 | 1 |
| Total | 400 | 100 |

### Preparation of Formulation 1

Three different batches of tablets are prepared using the three different batches of micronized 5-CNAC disodium, one tablet batch having an average 5-CNAC disodium particle size of 46 microns (Batch A), a second tablet batch having an average 5-CNAC disodium particle size of 6 microns (Batch B), and a third tablet batch having an average 5-CNAC disodium particle size of 35 microns (Batch C).

0.50 g of salmon calcitonin, pre-screened through a 40 mesh screen, 57 g of micronized 5-CNAC disodium salt, screened through a 35 mesh screen, and 10 g of Polyplasdone XL (crospovidone, NF, International Specialty Products, 1361 Alps Road, Wayne, N.J., 07470, USA) is combined in a 500 mL jar and is mixed using a Turbula mixer for 100 revolutions at a speed of 46 RPM. An additional 57 g of micronized 5-CNAC disodium salt, screened through a 35 mesh screen, and 36.75 g of Avicel PH 102® is added to the jar and mixed for 500 revolutions at a speed of 46 RPM. A further 36.75 g of Avicel PH 102® is added to the jar and is mixed for an additional 100 revolutions at a speed of 46 RPM. 4.0 g of magnesium stearate is screened into the jar using a 35 mesh screen and is blended for 1 minute at a speed of 46 RPM. The final blend is compressed into tablets using a Manesty B3B tablet press. The tablet weight is approximately 400 mg.

The bioavailability of the tablets created in this example may be tested as described in Example 13.

### Prophetic Example 13

### Primate Administration

The tablets are prepared as in Example 12 using three different batches of micronized 5-CNAC disodium, one tablet batch having an average 5-CNAC disodium particle size of 46 microns (Batch A), a second tablet batch having an average 5-CNAC disodium particle size of 6 microns (Batch B), and a third tablet batch having an average5-CNAC disodium particle size of 35 microns (Batch C). Each tablet contains 200 mg 5-CNAC disodium and 1 mg salmon calcitonin. The tablets prepared from each of the three different batches are administered to the same four Rhesus monkeys separately on different days as follows:

The Rhesus monkeys fast overnight prior to dosing and are restrained in chairs fully conscious, for the duration of the study period. One tablet from Batch A or Batch B or Batch C is administered to each monkey via a gavage tube followed by 10 mL of water.

Rhesus monkey blood samples are collected immediately before administration and at 0.25, 0.5, 0.75, 1,1. 5,2, 3,4, 5, and 6 hours after administration. A tablet from each of the remaining two tablet batches is dosed and blood samples are collected in a similar manner but on a separate day for each of the remaining tablet batches. Resulting plasma salmon calcitonin for each dose and for each monkey is determined by radioimmunoassay. For each monkey, the primate plasma salmon calcitonin (SCt) for one batch and one time period, mean plasma SCt concentrations for all monkeys for one batch and one time period, Standard Deviation (SD) of plasma SCt concentrations for one batch and one time period, and Standard Error of the Mean (SEM) for plasma SCt concentrations for all monkeys for one batch and one time period are calculated. The prophetic results are shown in the tables below.

BATCH A: AVERAGE 5-CNAC PARTICLE SIZE 46 MICROMETERS
Salmon Calcitonin (SCt) Plasma Concentrations [pg/mL]
(Single Oral Tablet (200 mg 5-CNAC + 1 mg SCt) to the Rhesus Monkey)

| Animal | Time [hours] | | | | | | | | | | |
|---|---|---|---|---|---|---|---|---|---|---|---|
| no. | 0 | 0.25 | 0.50 | 0.75 | 1 | 1.5 | 2 | 3 | 4 | 5 | 6 |
| 1 | 0.0 | 17.8 | 91.7 | 279.7 | 449.2 | 278.8 | 48.0 | 10.5 | 5.3 | 3.3 | 0.0 |
| 2 | 0.0 | 117.4 | 535.0 | 430.8 | 981.4 | 1718.0 | 2396.4 | 719.5 | 253.6 | 102.1 | 62.9 |
| 3 | 0.0 | 113.9 | 754.5 | 1502.0 | 2351.0 | 2066.0 | 2684.4 | 1310.0 | 649.6 | 280.6 | 156.5 |
| 4 | 0.0 | 46.0 | 127.0 | 425.5 | 765.8 | 1102.0 | 1599.0 | 1022.0 | 419.3 | 87.0 | 23.4 |
| Mean | 0.0 | 73.8 | 377.1 | 659.5 | 1136.9 | 1291.2 | 1682.0 | 765.5 | 332.0 | 118.3 | 60.7 |
| SD | 0.0 | 49.7 | 322.2 | 566.0 | 838.4 | 783.8 | 1182.1 | 558.1 | 271.6 | 116.6 | 68.9 |
| SEM | 0.0 | 24.9 | 161.1 | 283.0 | 419.2 | 391.9 | 591.0 | 279.0 | 135.8 | 58.3 | 34.5 |

Lower Limit of Quantification (LLOQ) = 2.5 pg/mL, concentrations below LLOQ were set to zero for Table 1

BATCH B: AVERAGE 5-CNAC PARTICLE SIZE 6 MICROMETERS
Salmon Calcitonin (SCt) Plasma Concentrations [pg/mL]
(Single Oral Tablet (200 mg 5-CNAC + 1 mg SCt) to the Rhesus Monkey)

| Animal | Time [hours] | | | | | | | | | | |
|---|---|---|---|---|---|---|---|---|---|---|---|
| no. | 0 | 0.25 | 0.50 | 0.75 | 1 | 1.5 | 2 | 3 | 4 | 5 | 6 |
| 1 | 0.0 | 265.6 | 315.8 | 245.6 | 357.2 | 1927.0 | 3010.0 | 863.2 | 139.4 | 48.5 | 20.8 |
| 2 | 0.0 | 607.0 | 777.0 | 1336.0 | 1602.0 | 4146.0 | 7521.0 | 2681.0 | 420.8 | 73.9 | 43.2 |
| 3 | 0.0 | 80.9 | 225.5 | 325.6 | 655.6 | 1478.0 | 3979.0 | 2775.0 | 520.2 | 91.5 | 41.3 |
| 4 | 0.0 | 286.4 | 155.3 | 237.7 | 241.0 | 269.7 | 294.2 | 321.0 | 179.8 | 67.5 | 13.6 |
| Mean | 0.0 | 310.0 | 368.4 | 536.2 | 714.0 | 1955.2 | 3701.1 | 1660.1 | 315.1 | 70.4 | 29.7 |
| SD | 0.0 | 218.5 | 280.2 | 534.7 | 617.2 | 1619.6 | 2986.3 | 1253.5 | 184.8 | 17.8 | 14.8 |
| SEM | 0.0 | 109.2 | 140.1 | 267.3 | 308.6 | 809.8 | 1493.1 | 626.7 | 92.4 | 8.9 | 7.4 |

Lower Limit of Quantification (LLOQ) = 2.5 pg/mL, concentrations below LLOQ were set to zero for Table 2

BATCH C: AVERAGE 5-CNAC PARTICLE SIZE 35 MICROMETERS
Salmon Calcitonin (SCt) Plasma Concentrations [pg/mL]
(Single Oral Tablet (200 mg 5-CNAC + 1 mg SCt) to the Rhesus Monkey)

| Animal | Time [hours] | | | | | | | | | | |
|---|---|---|---|---|---|---|---|---|---|---|---|
| no. | 0 | 0.25 | 0.50 | 0.75 | 1 | 1.5 | 2 | 3 | 4 | 5 | 6 |
| 1 | 0.0 | 36.1 | 94.7 | 428.0 | 739.4 | 2568.0 | 4025.0 | 1348.0 | 499.6 | 218.4 | 98.1 |
| 2 | 0.0 | 10.9 | 55.0 | 168.9 | 248.2 | 507.3 | 654.0 | 434.8 | 177.3 | 68.8 | 38.9 |
| 3 | 0.0 | 172.3 | 336.6 | 409.5 | 584.9 | 1487.0 | 2087.0 | 1479.0 | 162.0 | 52.0 | 17.2 |
| 4 | 0.0 | 7.9 | 46.9 | 208.1 | 390.1 | 1237.0 | 2347.0 | 1342.0 | 192.3 | 42.3 | 19.2 |
| Mean | 0.0 | 56.8 | 133.3 | 303.6 | 490.7 | 1449.8 | 2278.3 | 1151.0 | 257.8 | 95.4 | 43.4 |
| SD | 0.0 | 78.0 | 137.1 | 134.1 | 215.8 | 853.5 | 1382.1 | 481.6 | 161.7 | 82.7 | 37.8 |
| SEM | 0.0 | 39.0 | 68.6 | 67.1 | 107.9 | 426.7 | 691.1 | 240.8 | 80.8 | 41.4 | 18.9 |

Lower Limit of Quantification (LLOQ) = 2.5 pg/mL, concentrations below LLOQ were set to zero for Table 3

The compositions according to the instant invention allow considerably improved oral bioavailability of active agent. The improved bioavailability results in high in vivo concentrations of active agent, particularly calcitonin, being achieved via oral delivery, and in correlation to the particle sizes of 5-CNAC in the oral formulations of the examples.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited throughout this application, the disclosures of which are incorporated herein by reference in their entireties for all purposes.

1. Particles comprising a delivery agent and an active agent, wherein the particles have a median particle size of less than about 999 micrometers.

2. The particles of claim 1, wherein the particles have a median particle size of about 1 nanometer to about 999 micrometers.

3. The particles of claim 2, wherein the particles have a median particle size of about 1 to about 999 micrometers.

4. The particles of claim 2, wherein the particles have a median particle size of about 1 to about 999 nanometers.

5. The particles of claim 2, wherein the particles have a median particle size of about 45 to about 850 micrometers.

6. The particles of claim 2, wherein the particles have a median particle size of about 45 to about 150 micrometers.

7. The particles of claim 2, wherein the particles have a median particle size of about 150 to about 250 micrometers.

8. The particles of claim 2, wherein the particles have a median particle size of about 250 to about 425 micrometers.

9. The particles of claim 2, wherein the particles have a median particle size of about 425 to about 850 micrometers.

10. The particles of claim 2, wherein the particles have a median particle size of about 100 to about 1000 nanometers.

11. The particles of claim 10, wherein the particles have a median particle size of about 500 to about 1000 nanometers.

12. A pharmaceutical formulation comprising particles having a median particle size of less than about 999 micrometers, the particles comprising a delivery agent and an active agent.

13. The pharmaceutical formulation of claim 12, wherein the particles have a median particle size of about 1 nanometer to about 999 micrometers.

14. The pharmaceutical formulation of claim 13, wherein the particles have a median particle size of about 1 to about 999 micrometers.

15. The pharmaceutical formulation of claim 13, wherein the particles have a median particle size of about 1 to about 999 nanometers.

16. The pharmaceutical formulation of claim 13, wherein the particles have a median particle size of about 45 to about 850 micrometers.

17. The pharmaceutical formulation of claim 13, wherein the particles have a median particle size of about 45 to about 150 micrometers.

18. The pharmaceutical formulation of claim 13, wherein the particles have a median particle size of about 150 to about 250 micrometers.

19. The pharmaceutical formulation of claim 13, wherein the particles have a median particle size of about 250 to about 425 micrometers.

20. The pharmaceutical formulation of claim 13, wherein the particles have a median particle size of about 425 to about 850 micrometers.

21. The pharmaceutical formulation of claim 13, wherein the particles have a median particle size of about 100 to about 1000 nanometers.

22. The pharmaceutical formulation of claim 21, wherein the particles have a median particle size of about 500 to about 1000 nanometers.

23. A pharmaceutical formulation comprising a delivery agent and an active agent, wherein the delivery agent is in the form of particles having a median particle size of less than about 999 micrometers.

24. The pharmaceutical formulation of claim 23, wherein the delivery agent particles have a median particle size of about 1 nanometer to about 999 micrometers.

25. The pharmaceutical formulation of claim 23, wherein the active agent is in the form of particles, and the active agent particles have a median particle size of about 1 nanometer to about 999 micrometers.

26. The pharmaceutical formulation of claim 24, wherein the delivery agent particles have a median particle size of about 1 to about 999 micrometers.

27. The pharmaceutical formulation of claim 24, wherein the delivery agent particles have a median particle size of about 1 to about 999 nanometers.

28. The pharmaceutical formulation of claim 26, wherein the delivery agent particles have a median particle size of about 7 to about 16 micrometers.

29. The pharmaceutical formulation of claim 23, wherein the delivery agent particles are compressed to form micro-beads.

30. The pharmaceutical formulation of claim 29, wherein the micro-beads are coated with an active agent.

31. The pharmaceutical formulation of claim 30, wherein the active agent is insulin or heparin.

32. The pharmaceutical formulation of claim 29, wherein the micro-beads have a diameter of about 0.2 mm to about 2.0 mm.

33. The pharmaceutical formulation of claim 25, wherein the delivery agent particles and the active agent particles both have a median particle size of about 1 to about 999 micrometers.

34. The pharmaceutical formulation of claim 25, wherein the delivery agent particles and the active agent particles both have a median particle size of about 1 to about 999 nanometers.

35. A pharmaceutical formulation comprising a delivery agent and an active agent, wherein the active agent is in the form of particles having a median particle size of less than about 999 micrometers.

36. The pharmaceutical formulation of claim 35, wherein the active agent particles have a median particle size of about 1 nanometer to about 999 micrometers.

37. The pharmaceutical formulation of claim 35, wherein the active agent particles have a median particle size of about 1 to about 999 micrometers.

38. The pharmaceutical formulation of claim 17, wherein the active agent particles have a median particle size of about 1 to about 999 nanometers.

39-94. (canceled)
